(19) United States
(12) Patent Application Publication
(10) Pub. No.: US 2025/0340844 A1
(43) Pub. Date: Nov. 6, 2025

(54) LIVER SPHEROID DISEASE MODELS

(71) Applicant: Pandorum Technologies Private Limited, Bangalore (IN)

(72) Inventors: Tuhin BHOWMICK, New York, NY (US); Arun CHANDRU, Bangalore (IN); Wenson David Rajan KARUNAKARAN, Bangalore (IN); Nisha Palanisamy RAJENDRAN, Bangalore (IN)

(21) Appl. No.: 19/050,018

(22) Filed: Feb. 10, 2025

Related U.S. Application Data
(63) Continuation of application No. PCT/IN2023/050770, filed on Aug. 11, 2023.

(30) Foreign Application Priority Data
Aug. 11, 2022 (IN) 202241045831

(51) Int. Cl. C12N 5/071

(52) U.S. Cl. CPC C12N 5/0671; C12N 2500/36; C12N 2501/15; C12N 2513/00

(57) ABSTRACT

Disclosed is a method of inducing non-alcoholic steato-hepatitis in a multi-component liver spheroid. The method includes harvesting or reviving a cryopreserved sample of two or more distinct primary liver cell types and seeding a mixture of at least two or more distinct primary liver cell types to generate a multi-component liver spheroid. Disclosed are mono-component and multi component liver spheroid. The method further includes inducing steatosis in the multi-component liver spheroid through treatment of one or more steatosis inducers, inducing steatohepatitis in the multi-component liver spheroid through a combinatorial treatment of a mixture of the one or more steatosis inducers and one or more fibrosis inducers; and inducing fibrosis in the multi-component liver spheroid through treatment with one or more fibrosis inducers.

### CROSS-REFERENCE TO RELATED APPLICATIONS

This application is a continuation of International Application No. PCT/IN2023/050770, filed on Aug. 11, 2023, which claims the benefit of Indian Patent Application number 202241045831, filed Aug. 11, 2022 the entire contents of which are incorporated herein by reference.

### FIELD OF INVENTION

The present disclosure broadly relates to the field of in-vitro cell culture, and particularly discloses methods of generating liver spheroids.

### BACKGROUND OF INVENTION

Non-alcoholic fatty liver disease (NAFLD) is thought to be the leading cause of chronic liver disease worldwide. Currently there are no FDA approved medical therapies for NAFLD. A normal liver may progress through the different stages of NAFLD from steatosis to non-alcoholic steatohepatitis (NASH), and finally fibrosis. Although no FDA approved treatments exist, the field is actively searching to identify new targets and therapeutics to treat NAFLD.

Although historically many therapeutics have been screened using primary cells and cell lines in two-dimensional culture, these methods do not accurately depict the three-dimensional structure and architecture of the liver.

Liver organoids and liver spheroids attempt to closely mimic in vivo systems by having the formation of important three-dimensional tissue like architecture, heterogeneity of cells present, and the interactions/crosstalk between the different cell types. In addition, liver spheroids have been used to model a disease through different culturing method and different cocktails inducing agents. However, there remains a need for liver spheroid-based disease models such as for NAFLD and its various stages that are relatively simple to prepare and maintain on the one hand, and accurately reflect the in vivo disease state including recapitulating cellular architecture and gene expression patters of diseased liver when subjected to disease conditions on the other hand.

### SUMMARY

Advantageously, liver organoids and liver spheroids can be induced through cocktails of inflammatory cytokines, free fatty acids, and sugars to display phenotypes or markers of the different stages of NAFLD. Furthermore, these induced liver organoids or liver spheroids may be used to screen or test a variety of different compounds or therapeutics for effectiveness against one or more stages of NAFLD.

In an aspect of the present disclosure, a method of inducing non-alcoholic steatohepatitis within a multi-component liver spheroid may include providing two or more distinct liver cell types and seeding a mixture of at least two or more distinct primary liver cell types to generate a multi-component liver spheroid. Optionally, at least one of the two or more distinct liver cell types are human liver cells and/or are primary liver cells. The method may further include inducing steatosis in the multi-component liver spheroid through treatment of one or more steatosis inducers, inducing steatohepatitis in the multi-component liver spheroid through a combinatorial treatment of a mixture of the one or more steatosis inducers and one or more fibrosis inducers, and inducing fibrosis in the multi-component liver spheroid through treatment with one or more fibrosis inducers.

In an aspect of the present disclosure, the multi-component liver spheroid may be a bi-component liver spheroid consisting of a first type of liver cell and a second type of liver cell. The first type of liver cell may be hepatocytes and the second type of liver cell may be hepatic stellate cells.

In an aspect of the present disclosure, the multi-component liver spheroid may be comprised of three of more types of liver cells selected from the group consisting of primary human hepatocytes, hepatic stellate cells, Kupffer cells, and liver endothelial cells.

In an aspect of the present disclosure, the first type of liver cell and the second type of liver cell may be seeded at a cell count ratio within the range of about 50:50 to about 90:10, including the cell count ratio of the first type of liver cell to the second type of liver being about 70:30.

In an aspect of the present disclosure, a mixture of at least two or more distinct primary liver cell types may be used to generate a multi-component liver spheroid. The mixture may include seeding a total number of cells of about 750 cells to about 2000 cells per multi-component liver spheroid.

In an aspect of the present disclosure, the one or more steatosis inducers may comprise one or more free fatty acids. The one or more free fatty acids may comprise one or more free fatty acids selected from the group consisting of oleic acid, palmitic acid, lineolenic acid, and linoleic acid. In some variations, the one or more free fatty acids may comprise a mixture of two or more free fatty acids, where the mixture of the two or more free fatty acids comprise a ratio by weight of the two or more free fatty acids. The mixture of two or more free fatty acids may include a first free fatty acid and a second free fatty acid with the ratio by weight of the first fatty acid to the second fatty acid in the mixture may be about 2:1.

In an aspect of the present disclosure, the first fatty acid may be oleic acid, and the second fatty acid may be palmitic acid. The one or more fatty acids used to induce the multi-component liver spheroid may be at a final concentration within the range of about 100 μM to about 800 μM, including at the final concentration of about 600 μM.

In an aspect of the present disclosure, inducing steatosis in the multi-component liver spheroid through treatment of one or more steatosis inducers includes treating the multi-component liver spheroids for about 5 days to about 7 days including for about 6 days.

In an aspect of the present disclosure, the one or more fibrosis inducers may include one or more inflammatory cytokines. The one or more inflammatory cytokines may be selected from the group consisting of TGF-β1, IL-1β, TNFα, IL-6, IL-15, IL-17, and IL-18. In some variations, the one or more fibrosis inducers used to treat the multi-component liver spheroid are at a final concentration within the range of about 1 ng/mL to about 100 ng/mL including about 10 ng/ml to about 20 ng/mL.

In an aspect of the present disclosure, inducing steatohepatitis in the multi-component liver spheroids through a combinatorial treatment of a mixture of the one or more steatosis inducers and the one or more fibrosis inducers includes treating the multi-component liver spheroid with the mixture of one or more steatosis inducers and the one or more fibrosis inducers for about 24 hours to about 72 hours including for about 48 hours.

In an aspect of the present disclosure, inducing fibrosis in the multi-component liver spheroid through treatment with one or more fibrosis inducers includes removing the one or more steatosis inducers and treating the multi-component liver spheroid with the one or more fibrosis inducers for about 24 hours to about 72 hours including for about 48 hours.

In an aspect of the present disclosure, a multi-component liver spheroid may be produced including a bi-component liver spheroid. In some variations, a multi-component steatohepatic liver spheroid may be produced that is characterized by having one or more of, as compared to a healthy liver spheroid at least a two-log fold reduction in MT3, at least a two-log fold reduction in APOA4, and at least a two-log fold reduction in IGFBP1.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts a flow chart of exemplary methods of generating mono-component or multi-component liver spheroids and inducing steatosis, steatohepatitis, early-stage fibrosis, or late-stage fibrosis in the liver spheroids in accordance with embodiments of the disclosure.

FIG. 2 depicts a timeline of the exemplary methods inducing steatosis, steatohepatitis, early-stage fibrosis, or late-stage fibrosis in the liver spheroids produced in accordance with embodiments of the disclosure.

FIG. 3 depicts a flow chart of exemplary methods of generating mono-component and bi-component spheroids, inducing early-stage fibrosis, late-stage fibrosis, and NASH (steato-fibrosis) within bi-component spheroids produced in accordance with embodiments of the disclosure.

FIG. 4A depicts a representative confocal microscopy image of a mono-component liver spheroid produced in accordance with an embodiment of the disclosure that is immuno-stained for phalloidin (Scale bar 50 μm).

FIG. 4B depicts representative microscopy images of mono-component liver spheroids produced in accordance with an embodiment of the disclosure at day 7, day 11, day 15, and day 19 that are immuno-stained for functional markers (Scale bar 50 μm).

FIG. 4C illustrates immunofluorescence micrographs indicating PDGFR-β, α-SMA, vimentin staining in stellate cells on day 5 when grown in 2D cultured plate and on day 7 in bi-component liver spheroid culture (Scale bar 50 μm for 2D images; scale bar 100 μm for liver spheroids micrograph).

FIG. 4D depicts representative microscopy images of bi-component liver spheroids produced in accordance with embodiments of the disclosure at day 7, day 15 or day 21. Representative images include phase-contrast to demonstrate liver tissue morphology, Hematoxylin and Eosin (H&E) to demonstrate microarchitecture, MTS to demonstrate collagen deposition, and immunofluorescence staining to demonstrate hepatocyte (CYP3A4) and human stellate cell (Vimentin) specific protein markers.

FIG. 5A depicts a schematic of inducing a steatotic phenotype in mono-component spheroids in accordance with embodiments of the disclosure.

FIGS. 5B-5C depict representative images of mono-component spheroids (Trial 1 and Trial 2) produced in accordance with embodiments of the disclosure accumulating intracellular lipids by Nile Red staining during steatosis.

FIGS. 5D-5E Depicts histology (H&E stained) micrographs of control and FFA treated PHH spheroids indicating the nuclear rearrangement (black arrows) and hepatocytes ballooning (Scale bar 50 μm) in accordance with embodiments of the disclosure.

FIG. 5F illustrates a schematic of inducing a fibrotic phenotype in bi-component liver spheroids produced in accordance with embodiments of the disclosure.

FIG. 5G depicts representative microscopic images of bi-component liver spheroids immuno-stained with Masson's Trichrome staining for collagen deposition after 2, 4, and 7 days of fibrotic stimuli in accordance with embodiments of the disclosure.

FIG. 5H illustrates a Bar graph showing the secreted albumin levels via ELISA after 2, 4 and 7 days of TGF-β1 treatment in PHH and PSC spheroids vs control in accordance with embodiments of the disclosure.

FIG. 6A depict representative microscopy images of immunofluorescence staining of CYP3A4 in bi-component liver spheroids generated in trial 1 after 2 days and 7 days of treatment with TGF-β1 in accordance with embodiments of the disclosure.

FIG. 6B depicts representative microscopy images of immunofluorescence staining of CYP3A4 in bi-component liver spheroids generated in trial 2 after 4 days and 7 days treatment with TGF-β1 in accordance with embodiments of the disclosure.

FIG. 6C depicts disease induction in mono-component liver spheroids. The Bar graph shows the secreted albumin levels assessed via ELISA on day 7 of FFA treatment in PHH spheroids vs control. (n=3, *p<0.05, ns not significant), in accordance with embodiments of the disclosure.

FIGS. 6D and 6E depict disease induction in bi-component liver spheroids. FIG. 6D shows immunofluorescence micrographs and FIG. 6E shows bar graph of quantified relative fluorescence intensity indicating variance in fibronectin expression in fibrotic liver spheroids in accordance with embodiments of the disclosure. (scale bar 100 μm)

FIGS. 6F and 6G depict disease induction in bi-component liver spheroids. FIG. 6F shows micrographs obtained after SDS PAGE western blot assay showing the protein bands for α-SMA (band observed at 42 kDA) and fibronectin (band observed at 263 kDa) after 4 and 7 days TGF-β1 treatment in PHH and PSC spheroids vs control. GAPDH (band observed at 37 kDa) as loading control. FIG. 6G shows bar graphs indicating quantified fold change in α-SMA and fibronectin expression (normalised to control) via densitometry analysis of western blotting assay micrographs, in accordance with embodiments of the disclosure. (n=3)

FIGS. 7A-7B depicts portions of a heat map of gene expression of early-stage fibrosis induced liver spheroids produced in accordance with embodiments of the disclosure compared to published human gene expression data related to liver cirrhosis, NASH, and liver failure.

FIG. 8 depicts a heat map of gene expression of late-stage fibrosis induced liver spheroids produced in accordance with embodiments of the disclosure compared to published human gene expression data related to liver cirrhosis, NASH, and liver failure.

FIGS. 9A-9D depict heat maps of gene expression data of genes within key pathways in healthy liver spheroids and NASH induced liver spheroids produced in accordance with embodiments of the disclosure.

FIGS. 10A-10B depict heat maps of the gene expression from NASH induced liver spheroids produced in accordance with embodiments of the disclosure compared to published human gene expression profiles based on the livers of NASH patients.

FIGS. 11A-11B depict heat maps of the gene expression from NASH induced liver spheroids produced in accordance with embodiments of the disclosure compared to published human gene expression profiles based on the livers of patients suffering from NASH or other liver conditions.

FIG. 12 depict the data for Enriched Biological Processes from differentially expressed genes FFA+TGF-β1 treated groups compared to Healthy Control groups. (A) The bar plot depicts the significantly enriched relevant gene ontologies (Biological processes) from up regulated genes in Diseased (FFA+TGF-β1 treated) sample, in accordance with embodiments of the disclosure.

(B) Bar plot captures significantly enriched relevant gene ontologies (Biological processes) from down regulated genes in Diseased (FFA+TGF-β1 treated) sample

“DAVID” tool was used for the GO ontology enrichment analysis, in accordance with embodiments of the disclosure.

FIG. 13 depict the data for Enriched Biological Processes from differentially expressed genes FFA+TGF-β1 treated groups compared to Healthy Control groups. (A) The bar plot depicts the significantly enriched relevant gene ontologies (Biological processes) from up regulated genes in Diseased (FFA+TGF-β1 treated) sample, in accordance with embodiments of the disclosure.

(B) Bar plot captures significantly enriched relevant gene ontologies (Biological processes) from down regulated genes in Diseased (FFA+TGF-β1 treated) sample

“DAVID” tool was used for the GO ontology enrichment analysis, in accordance with embodiments of the disclosure.

FIG. 14 (A to F) depict Heatmap representation of constituent genes of the Enriched Biological Processes from up regulated genes in Diseased (FFA+TGF-β1 treated) sample, compared to Healthy control (For FIG. 12), in accordance with embodiments of the disclosure.

FIG. 14 (G to O) depict heatmap representation of constituent genes of the Enriched Biological Processes from down regulated genes in Diseased (FFA+TGF-β1 treated) sample, compared to Healthy control. (For FIG. 12), in accordance with embodiments of the disclosure.

FIG. 14P depicts Heatmap representation of constituent genes known to be involved in NASH/Fibrosis (For FIG. 12), in accordance with embodiments of the disclosure.

FIG. 15 (A to C) depict Heatmap representation of constituent genes of the Enriched Biological Processes from up regulated genes in Diseased (FFA+TGF-β1 treated) sample, compared to Healthy control (For FIG. 13), in accordance with embodiments of the disclosure.

FIG. 15 (D to M) depict Heatmap representation of constituent genes of the Enriched Biological processes from down regulated genes in Diseased (FFA+TGF-β1 treated) sample, compared to Healthy control (For FIG. 13), in accordance with embodiments of the disclosure.

FIG. 15N depicts Heatmap representation of constituent genes known to be involved in NASH/Fibrosis (For FIG. 13), in accordance with embodiments of the disclosure.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any or all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “three-dimensional culture” or “3D culture” refers to a system of culturing the cells in-vitro in which the biological cells are allowed to grow and interact with their surroundings in all the three dimensions.

The term “two-dimensional culture” or “2D culture” refers to the method of culturing the cells substantially as a monolayer on a surface by which the biological cells are able to interact with their surroundings in two dimensions, although some negligible three-dimensional interaction may occur.

The term “NASH” refers to non-alcoholic steato-hepatitis that contains aspects of steatosis and fibrosis and the term “steato-fibrosis” could be used interchangeably with steato-hepatitis in this application.

### Non-Alcoholic Fatty Liver Disease

Non-alcoholic fatty liver disease (NAFLD) is a term used to describe a range of diseased conditions of the liver. The liver's functions in the body include filtering blood, metabolizing drugs, production of proteins for blood plasma, regulating amino acid levels in the blood, production of proteins important for carrying fats throughout the body, production of bile and helping cleanse the body of toxic substances. The liver can acquire an abundance of fat cells, potentially severely, even irreversibly, impairing the functions of the liver. Consistent injury to the liver can progress the liver through different stages of NAFLD, including steatosis (non-alcoholic fatty liver or NAFL) that is characterized by accumulation of fat in the liver and steato-hepatitis (non-alcoholic steato-hepatitis or NASH) in which fat accumulation is coupled with inflammation and fibrosis. NASH can further induce or progress to fibrosis. The stages of NAFLD can be linear where progress occurs over time from normal liver tissue to fibrotic liver tissue, but the stages of NAFLD can also be fluid, with acute injury to the liver tissue bypassing earlier states and progressing to most severe stages (e.g., fibrosis).

Steatosis is characterized by intracellular accumulation of lipids and triglycerides. Further injury to liver tissue can lead to steato-hepatitis characterized by activation of Kupffer cells and secretion of key inflammatory cytokines such as IL-6 or TNF-α. Steato-hepatitis may progress to include fibrosis where key physiological markers include activation of stellate cells, presence of collagen and other components of the extracellular matrix. As NAFLD progresses from a normal liver to fibrosis, the complexity of the disease increases. Currently there are no FDA approved therapeutics for NAFLD, thus it is important to develop models that accurately model disease including the different stages of NAFLD. Historically, two-dimensional (2D) (e.g., monolayer cultures) cell cultures were used to interrogate different therapeutics in NAFLD. However, 2D cell culture lacks liver architecture and other complex cell-cell interactions that may be required for resolving some states of NAFLD. Liver spheroids, which are three dimensional (3D) structures generated from primary liver cells, may under certain circumstances more accurately mimic liver cell makeup and architecture compared to 2D cell cultures.

The liver is composed of four major cell types: hepatocytes, hepatic stellate cells, Kupffer cells, and sinusoidal endothelial cells with hepatocytes most responsible for drug and lipid metabolism, as well as the secretion of coagulation and complement factors. Hepatic stellate cells are responsible for lipid storage and when injured, can play a role in liver fibrosis development. There is a need for developing liver spheroids that accurately recapitulate in vivo cellular architecture and gene expression patterns of healthy liver when healthy and accurately recapitulate in vivo cellular architecture and gene expression patterns of diseased liver when subjected to disease conditions. Such liver spheroid-based disease models can be used to test therapeutics compounds to determine which, if any, may be effective at treating liver diseases in vivo.

As used herein, a liver spheroid refers to a cluster of liver cells grown in culture. Generally, when liver cells are grown in culture under conditions where the liver cells do not attach to a surface, (e.g., if the cells are grown in a well with ultra-low attachment surfaces) the liver cells adhere to each other and self-aggregate into a spheroid. The liver cells used to grow a spheroid may be a liver cell line or primary liver cells such as hepatocytes or hepatic stellate cells.

### Methods of Generating Liver Spheroids and Inducing Disease Phenotypes Therein

Methods for inducing steatosis, non-alcoholic steatohepatitis (NASH), early-stage fibrosis, or late-stage fibrosis generally include culturing primary liver cells to generate liver spheroids and using a cocktail comprising one or more disease inducers to induce one or more desired disease states (e.g., steatosis, NASH, early-stage fibrosis, or late-stage fibrosis) within the liver spheroids. The method for inducing non-alcoholic steatohepatitis in a mono-component liver spheroid may generally include steps of seeding a distinct primary liver cell type to generate a mono-component liver spheroid, and inducing a disease state. The method for inducing non-alcoholic steatohepatitis in a multi-component liver spheroid may generally include steps of preparing mixture of two or more distinct primary liver cell types, seeding the mixture of two or more distinct primary liver cell types to generate a multi-component liver spheroid, and inducing a disease state. In some variations, the disease state may be steatosis induced by treatment with one or more steatosis inducers. In some variations, the disease state may be steatohepatitis induced through a combinatorial treatment with one or more steatosis inducers and one or more fibrosis inducers. In some variations, the disease state may be fibrosis induced by treatment with one or more fibrosis inducers.

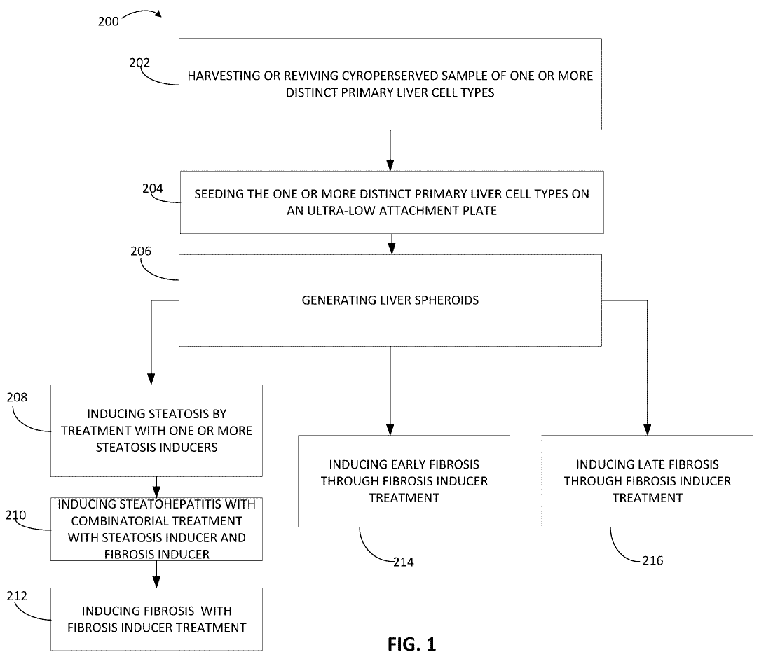

FIG. 1 depicts a flow chart of an exemplary method (200) of generating mono-component or multi-component liver spheroids and inducing steatosis, non-alcoholic steatohepatitis (NASH), early-stage fibrosis, or late-stage fibrosis in the liver spheroids. Mono-component liver spheroids are liver spheroids that comprise only one primary liver cell type while multi-component liver spheroids are liver spheroids that comprise more than one primary liver cell type. In some variations, multi-component liver spheroids include bi-component liver spheroids, tri-component liver spheroids, quad-component liver spheroids, or the like. In some variations, the liver spheroid may be a multi-component liver spheroid comprising two or more liver cell types selected from the group consisting of: hepatocytes, hepatic stellate cells, Kupffer cells, and endothelial cells. In some variations, the liver spheroid may be a bi-component liver spheroid consisting or essentially consisting of hepatocytes of hepatic stellate cells.

In exemplary embodiments, a method (200) includes harvesting or reviving one or more cryopreserved samples of one or more distinct primary liver cell types (202). Harvesting or reviving one or more cryopreserved samples of the one or more distinct primary liver cell types may include reviving one or more cryopreserved samples in 2D culture for at least about 16 hours. Reviving one or more cryopreserved samples of the one or more distinct primary liver cell types may include growing the one or more cryopreserved samples of the one or more distinct primary liver cell types in culture until each distinct cell type reach a confluence of about 70% to about 90%. In some variations, the one or more distinct primary liver cell types may include one or more of the following: hepatocytes, hepatic stellate cells, Kupffer cells, and endothelial cells.

The method (200) further includes seeding the one or more distinct primary liver cell types on an ultra-low attachment plate (204). In some variations for producing mono-component liver spheroids, seeding the one or more distinct primary liver cell types may comprise seeding primary human hepatocytes on the ultra-low attachment plate. In some variations for producing bi-component liver spheroids, seeding the one or more distinct primary liver cell types may comprise seeding a first primary liver cell type and a second primary liver cell type on the ultra-low attachment plate, wherein the first primary liver cell type is primary human hepatocytes, and the second primary liver cell type is primary human hepatic stellate cells. In some variations, seeding the one or more distinct primary liver cell types on an ultra-low attachment plate may include seeding a first primary liver cell type, a second primary liver cell type, optionally a third primary liver cell type, and optionally a fourth primary liver cell type. In some variations, seeding the one or more distinct primary liver cell types includes seeding about 750 to about 2000 total cells per well in a 96-well ultra-low attachment plate.

In some variations, the mixture of the first primary liver cell type and the second primary liver cell type may comprise a specific ratio of cell counts within the mixture. For example, the ratio of the first primary liver cell type to the second primary liver cell type may be a specific cell count ratio within the range of about 1:1, 1.5:1, 2:1, 2.33:1, 2.5:1, 3:1, 3.5:1, 5:1, 7.5:1, 9:1, 10:1, 70:30, 90:10, or the like, wherein the first primary liver cell type is at least equal to if not greater than the second primary liver cell type. Seeding the one or more distinct primary liver cell types may comprise seeding a mixture of the first primary liver cell type and the second primary liver cell type having a total cell count per well of about 750 to about 2000 at any of the specific ratios described above. In some variations, each well may be a well in a 96-well plate. For example, seeding a mixture of a first primary liver cell type and a second primary liver cell type at a ratio of 70:30 and a total cell number per well of about 2000 includes seeding about 1400 first primary liver cell type and about 600 second primary liver cell type per well on an ultra-low attachment plate.

The method (200) further includes generating the liver spheroids (206). Generating the liver spheroids may comprise growing the seeded one or more primary liver cell types in culture for a duration of between about 5 days and about 8 days, or about 7 days. In variations where only primary human hepatocytes are seeded on the ultra-low attachment plate and allowed to grow, mono-component liver spheroids will be generated. In variations where two distinct liver cell types are seeded on the ultra-low attachment plate and allowed to grow, bi-component liver spheroids are generated. In variations where three or more distinct liver cell types are seeded on the ultra-low attachment plate, multi-component liver spheroids will be generated. In some variations, generating the liver spheroids may include changing the culture media after about 5 days without disturbing the liver spheroids. In some variations, generating the liver spheroids may include, after allowing the seeded cells to grow for 5 days in culture, placing the ultra-low attachment plate containing the liver spheroids in an orbital shaker shaking at a rate of greater than 100 rpm for a period of time of between about 2 days and about 17 days including during early-stage fibrosis, late-stage fibrosis, or disease state induction.

In some variations, after the liver spheroids are generated in step (206), the spheroids may be treated with appropriate inducers to induce a disease state, which may be one of NASH, early-stage fibrosis, and late-stage fibrosis. Treatment of the spheroids generated in step (206) to induce NASH is disclosed herein below with respect to steps (208), (210), and (212). Treatment of the spheroids generated in step (206) to induce early-stage (or mild) fibrosis is disclosed hereinbelow with respect to step (214). Treatment of the spheroids generated in step (206) to induce early-stage (or mild) fibrosis is disclosed hereinbelow with respect to step (216). In some variations, the treatment of the spheroids with the appropriate inducer may be initiated at between 5 days and 10 days after seeding, between 6 days and 8 days after seeding, 6 days after seeding, 7 days after seeding, or 8 days after seeding.

### NASH Induction

A non-alcoholic steatohepatitis (NASH) phenotype comprises a combination of steatosis and fibrosis phenotypes. Furthermore, NASH demonstrates a couple signature characteristics including intracellular lipid accumulation, fibrosis, and inflammation. In some variations, to induce a NASH phenotype in liver spheroids, the steatosis phenotype may be induced first in the liver spheroids, followed by induction of the fibrotic phenotype. In some variations, the liver spheroids in which NASH is induced in accordance with steps (208), (210), and (212), may be bi-component spheroids, in which each spheroid consists or substantially consists of hepatocytes and hepatic stellate cells, optionally seeded at a cell count ratio of 70:30 (hepatocytes to hepatic stellate cells). To induce NASH, the method (200) may include inducing steatosis through treatment of the liver spheroids with one or more steatosis inducers (208). Treatment of the liver spheroids with the one or more steatosis inducers may include removing the media within the ultra-low attachment plate without disrupting the spheroids and replacing with media containing the one or more steatosis inducers. Inducing steatosis through treatment of the liver spheroids with one or more steatosis inducers may include contacting the liver spheroids with the one or more steatosis inducers, whereby inducing steatosis.

In some variations, the one or more steatosis inducers may be a free fatty acid (FFA) composition comprising one or more free fatty acids selected from the group consisting of oleic acid, palmitic acid, stearic acid, propinoic acid, butyric acid, valeric acid, nervonic acid, erucic acid, cicosatrienoic acid, cicosenoic acid, hypogeic acid, elaidic acid, lineolenic acid, linoleic acid, cicosapentaenoic acid, docosahexaenoic acid, arachidonic acid, docosapentaenoic acid, glutamic acid including monosodium glutamate and all variants or derivatives therein. The FFA composition may be administered to the liver spheroids at a final concentration in the culture medium at a range of between about 100 μM and about 1 mM, between about 200 M and about 800 μM, between about 300 μM and about 750 μM, between about 500 μM and about 700 μM, or the like. In some variations, where the FFA composition comprises a mixture of two or more steatosis inducers each of the free fatty acids may be present in the FFA composition in a defined ratio, optionally by weight. For example, in a case where the FFA composition comprises two free fatty acids, the defined ratio may include about 1:1, 1.5:1, 2:1, 2.5:1, 3:1, 3.5:1, 4:1, 4.5:1, 5:1, 10:1, 20:1, 50:1, 100:1, 200:1, 500:1, 1000:1 or the like. It can be appreciated that when a mixture of two or more steatosis inducers are used at a specific ratio by weight to induce steatosis in the liver spheroids, the mixture of the two or more steatosis inducers may still be used at a final concentration within the range of about 100 μM to about 1 mM.

The liver spheroids may be treated with the one or more steatosis inducers starting from between about 6 days and about 8 days after seeding, or about 7 days after seeding. The liver spheroids may be treated with the one or more steatosis inducers for a duration of about 4 days to about 8 days, about 4 days, about 5 days, about 6 days, about 7 days, or about 8 days. Treating the liver spheroids with steatosis inducers for about 4 days to about 8 days may comprise changing the media containing the steatosis inducers after every other day without disrupting the liver spheroids.

In some variations, the FFA composition may comprise or consist of oleic acid and palmitic acid at a ratio of about 2:1 by weight and administered to the liver spheroids at a final concentration of about 600 μM, starting from day 7 after seeding.

Following induction of steatosis in liver spheroids through treatment with steatosis inducers (208), the method (200) further includes inducing steatohepatitis in the liver spheroids through a combinatorial treatment of a mixture of one or more steatosis inducers and one or more fibrosis inducers (210). Inducing steatohepatitis in the liver spheroids through a combinatorial treatment of a mixture of one or more steatosis inducers and one or more fibrosis inducers includes contacting the liver spheroids with the combination of the one or more steatosis inducers and the one or more fibrosis inducers, whereby inducing steatosis. The combinatorial treatment may have a duration of between about 24 hours and about 72 hours, (including all values and sub-ranges therein), or about 48 hours. In some variations, the combinatorial treatment may include replacing the media containing only the one or more steatosis inducers from step (208) with media containing a mixture of the one or more steatosis inducers and the one or more fibrosis inducers. In some variations, the one or more steatosis inducers used to induce steatohepatitis may be the same steatosis inducers as described above or may be different steatosis inducers. In some variations, the one or more fibrosis inducers may include one or more inflammatory cytokines, inflammation activators including lipopolysaccharide (LPS), one or more signaling molecules, or one or more growth factors. In some variations, the one or more inflammatory cytokines may be selected from the group consisting of: TGF-β1, IL-1β, TNFα, IL-6, IL-15, IL-17, and IL-18. In some variations, the one or more growth factors may comprise connective tissue growth factor (CTGF) and/or platelet derived growth factor (PDGF). The one or more fibrosis inducers may be used at a final concentration of between about 1 ng/mL and about 200 ng/mL, or between about 10 ng/ml and about 30 ng/mL.

Generally, the transformation from steatosis to fibrosis in native liver tissue in vivo can take years of constant injury. However, in culture, this timeline may be accelerated. The method (200) may include inducing fibrosis in the liver spheroids through a continued treatment with one or more fibrosis inducers (212). Inducing fibrosis in the liver spheroids through a continued treatment with one or more fibrosis inducers includes contacting the liver spheroids with the one or more fibrosis inducers, whereby inducing fibrosis. Inducing fibrosis in the liver spheroids through a continued treatment with one or more fibrosis inducers may include accelerating of the induction of fibrosis in the liver spheroids through a continued treatment with one or more fibrosis inducers (212). Inducing fibrosis in the liver spheroids through a continued treatment with one or more fibrosis inducers includes removing the media containing the mixture of the one or more steatosis inducers and the one or more fibrosis inducers from step (210) and replacing with media containing the one or more fibrosis inducers, but not the one or more steatosis inducers. In some variations, the one or more fibrosis inducers used in the mixture of steatosis inducers and the one or more fibrosis inducers may be the same one or more fibrosis inducers used in the acceleration of fibrosis induction. In some variations, inducing fibrosis in the liver spheroids through a continued treatment with one or more fibrosis inducers may comprise treating the liver spheroids with the one or more fibrosis inducers for a duration of between about 24 hours and about 72 hours, (including all values and sub-ranges therein), or about 48 hours. In some variations of the method (200), the steps of inducing steatosis through treatment of the liver spheroids with one or more steatosis inducers (208), inducing steatohepatitis through a combinatorial treatment of a mixture of one or more steatosis inducers and one or more fibrosis inducers of the liver spheroids (210), and accelerating induction of fibrosis in the liver spheroids through a continued treatment with one or more fibrosis inducers (212) may all take place within an orbital shaker shaking at a rate of greater than 100 rpm for a period of time of about 7 days to about 10 days.

### Early-Stage Fibrosis

To induce early-stage fibrosis within the liver spheroids, following the step of generating liver spheroids (208), the method (200) may include inducing early-stage fibrosis through treatment with one or more fibrosis inducers (214). In some variations, the liver spheroids may be mono-component liver spheroids, bi-component liver spheroids, or multi-component liver spheroids. Treatment with the one or more fibrosis inducers may be initiated at between 5 days and 10 days after seeding, between 6 days and 8 days after seeding, 6 days after seeding, or 7 days after seeding. In some variations, the liver spheroids may be treated with the one or more fibrosis inducers for between about 2 days and about 5 days, (including all values and sub-ranges therein), or about 4 days. Inducing early-stage fibrosis through treatment with one or more fibrosis inducers may include replacing the media with media containing one or more fibrosis inducers without disturbing the liver spheroids. The one or more fibrosis inducers may include one or more signaling molecules, one or more growth factors, or one or more inflammatory cytokines including a mixture thereof. For example, the one or more fibrosis inducers may include a mixture of one or more growth factors and one or more inflammatory cytokines. The one or more inflammatory cytokines may include a mixture of two or more inflammatory cytokines. The inflammatory cytokines may be selected from the group of: TGF-β1, IL-1β, TNFα, IL-6, IL-15, IL-17, and IL-18. The one or more growth factors may include connective tissue growth factor (CTGF), platelet derived growth factor (PDGF), or the like. The one or more fibrosis inducers may be used at a final concentration within the range of about Ing/mL to about 100 ng/mL including about 10 ng/mL.

### Late-Stage Fibrosis

To induce late-stage fibrosis within the liver spheroids, following the step of generating liver spheroids (208), the method (200) may include inducing late-stage fibrosis through treatment with one or more fibrosis inducers (216). In some variations, the liver spheroids may be mono-component liver spheroids, bi-component liver spheroids, or multi-component liver spheroids. Inducing late-stage fibrosis through treatment with one or more fibrosis inducers may include an extended treatment of the liver spheroids with the one or more fibrosis inducers that is longer in duration compared to the induction of early-stage fibrosis (214). For example, the liver spheroids may be treated with the one or more fibrosis inducers for between about 5 days and about 8 days (including all values and sub-ranges therein), or about 7 days.

Treatment with the one or more fibrosis inducers may be initiated at between 5 days and 10 days after seeding, between 6 days and 8 days after seeding, 6 days after seeding, 7 days after seeding, or 8 days after seeding. Inducing late-stage fibrosis through treatment with the one or more fibrosis inducers may include replacing the media with media containing the one or more fibrosis inducers without disturbing the liver spheroids. The one or more fibrosis inducers may include one or more signaling molecules, one or more growth factors, or one or more inflammatory cytokines including a mixture thereof. For example, the one or more fibrosis inducers may include a mixture of one or more growth factors and one or more inflammatory cytokines. The one or more inflammatory cytokines may include a mixture of two or more inflammatory cytokines. The inflammatory cytokines may be selected from the group of: TGF-β1, IL-1β, TNFα, IL-6, IL-15, IL-17, and IL-18. The one or more growth factors may include connective tissue growth factor (CTGF), platelet derived growth factor (PDGF), or the like. The one or more fibrosis inducers may be used at a final concentration within the range of about Ing/mL to about 100 ng/ml including about 10 ng/ml to about 20 ng/mL.

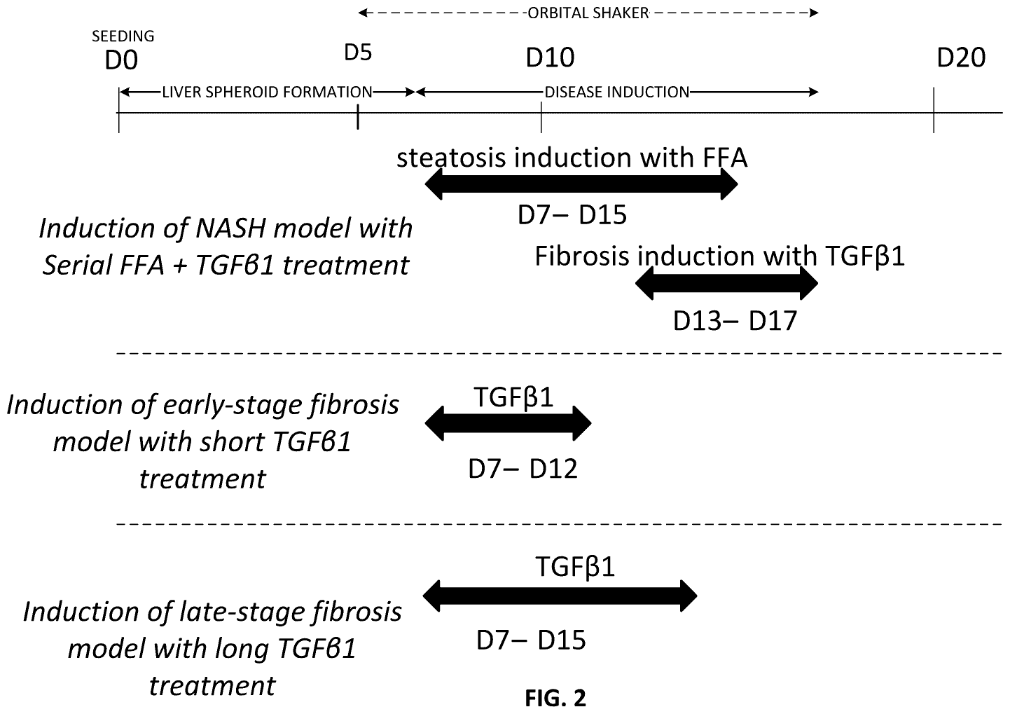

FIG. 2B depicts a schematic of a timeline generally followed in the method depicted in FIG. 2A that includes inducing steatosis, steatohepatitis, or fibrosis in the liver spheroids. As described above, one or more types of healthy primary liver cells may be seeded on ultra-low attachment plates to form liver spheroids on DO (day of seeding). The healthy primary liver cells may be thawed from cryopreserved samples and passaged until a desired concentration of healthy primary liver cells are in culture. Alternatively, the healthy primary liver cells may be harvested from primary samples. In some variations, to generate mono-component liver spheroids, primary human hepatocytes may be seeded at a density of about 750 to about 2000 cells per well in a 96-well ultra-low attachment plate at DO. In some variations, to generate bi-component liver spheroids, primary human hepatocytes and primary hepatic stellate cells may be seeded at a ratio of 70:30 and a density of up to about 2000 cells per well on the ultra-low attachment plate at DO. At D5 (day 5 after seeding), during formation of the liver spheroids, the ultra-low attachment plates containing the liver spheroids may be placed within an orbital shaker shaking at a rate of greater than 100 rpm during the rest of liver spheroid formation and during disease induction. After the formation of mono-component or bi-component liver spheroids at approximately D7 (day 7 after seeding), steatosis, steatohepatitis, early-stage fibrosis, or late-stage fibrosis may be induced in the liver spheroids. For example, steatosis (leading to intracellular lipid accumulation) may be induced through treatment with free fatty acids starting from D7 and continuing up to about D15. In inducing steatohepatitis (intracellular lipid accumulation with inflammation and fibrosis), the liver spheroids may be treated with free fatty acids from about D7 to about D13, wherein one or more fibrosis inducers may be added to the liver spheroids from D13-D15. At D15, free fatty acid treatment may be removed from the liver spheroids while treatment with the one or more fibrosis inducers continues from D15-D17.

To induce early-stage fibrosis in the mono-component or bi-component liver spheroids, at D7, liver spheroids may be subjected to treatment with one or more fibrosis inducers for about 5 days, from about D7 to about D12. The one or more fibrosis inducers used to induce early-stage fibrosis may include TGF-β1. To induce late-stage fibrosis in the mono-component or bi-component liver spheroids, at D7, liver spheroids are subjected to treatment with one or more fibrosis inducers for about 8 days, from about D7 to about D15. The one or more fibrosis inducers used to induce late-stage fibrosis may include TGF-1.

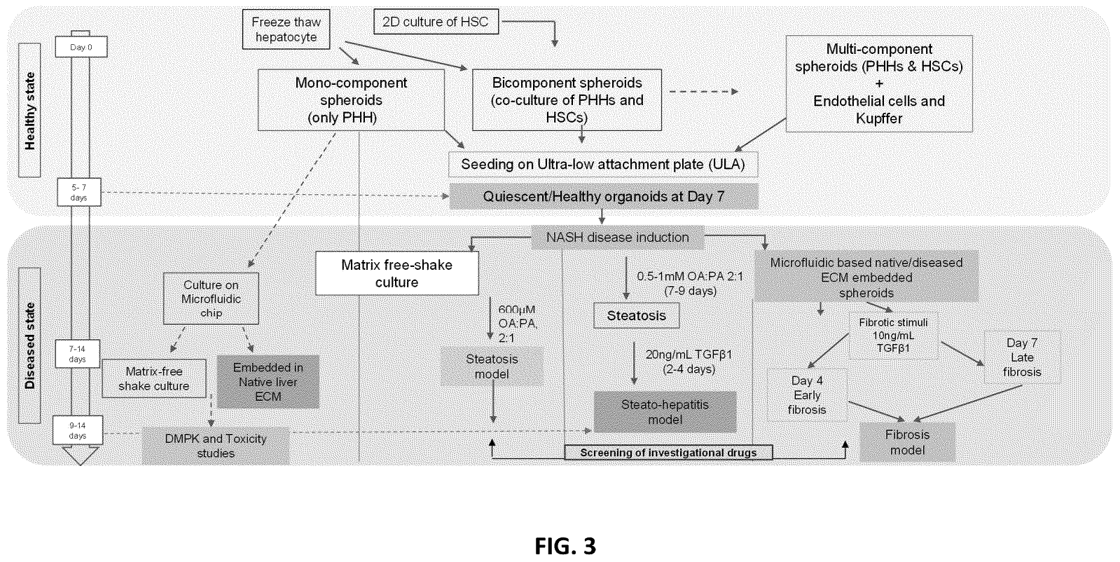

FIG. 3 depicts a flow chart of some methods of generating mono-component liver spheroids, by way of example with primary human hepatocytes (PHH), bi-component liver spheroids, by way of example with PHHs and hepatic stellate cells (HSCs), or multi-component liver spheroids, along with induction of early-stage fibrosis, late-stage fibrosis or NASH. The flow chart is broken up into an upper section (“Healthy state”) and a lower section (“Diseased state”) and generally flows from the upper section to the lower section. Following this flow chart may provide general direction as to generating healthy mono-component or multi-component liver spheroids. Progressing into the lower section may provide general direction to inducing disease within the liver spheroids. For example, healthy multi-component liver spheroids may be induced with a steatosis phenotype to generate a steatosis model. After generating healthy mono-component liver spheroids, the mono-component liver spheroids may be placed in a shake culture (e.g., orbital shaker) that is lacking in matrix proteins. As described in the method above, the one or more primary liver cell types may be seeded on an ultra-low attachment plate to generate the liver spheroids where the liver spheroids remain during disease induction. The liver spheroids may be placed within a shake culture to ensure diffusion of nutrients, oxygen, and disease inducers may reach the core of the liver spheroids. However, the liver spheroids may also be cultured on a microfluidic chip or in a microfluidic device. Advantageously, culturing liver spheroids on a microfluidic chip or in a microfluidic device provides the liver spheroids with a micro-physiological environment with continuous flow of media mimicking blood circulation. Furthermore, the liver spheroids may also be cultured on native extracellular matrix (ECM) to provide liver cell-ECM interactions. In some variations, the liver spheroids may be cultured on native ECM during generation of liver spheroids, during induction of the disease phenotypes (e.g., steatosis, steato-hepatitis, early-stage fibrosis, or late-stage fibrosis), or the combination thereof. Culturing liver spheroids on native ECM during induction of disease phenotypes may provide key liver ECM interactions during progression of disease. Furthermore, in some variations, liver spheroids may be cultured on ECM from diseased patients. For example, healthy liver spheroids may be cultured on ECM from NASH patients while the liver spheroids may then be induced to have a NASH phenotype, providing liver cell-diseased ECM interactions. In some variations, any of these culture methods may be combined to maximally reproduce the native liver microenvironment. For example, bi-component liver spheroids may be cultured on fibrotic ECM on a microfluidic chip while fibrosis is induced within the bi-component liver spheroids. After generation of healthy or diseased induced liver spheroids, the liver spheroids may be used in screening of investigational drugs. For example, the fibrotic induced liver spheroids may be used to screen for drugs that may partially or completely reverse the phenotype or genotype of fibrosis in the fibrotic induced liver spheroids. The NASH induced liver spheroids may be used to screen for drugs that may partially or completely reverse the phenotype or genotype of NASH in the NASH induced liver spheroids. Likewise, the disease induced liver spheroids may be used in drug metabolism and/or pharmacokinetics studies to further characterize new or existing drugs.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

For the purpose of the present disclosure, primary human hepatocytes (PHHs) or primary hepatic stellate cells (HSCs) are derived from the native liver tissue. In some variations, PHHs or HSCs were acquired from commercial manufacturers after being screened for non-desired characteristics or qualities. For example, non-desired characteristics of liver tissue donors may include below the age of about 5 and above the age of about 40, a history of heavy alcohol usage, and a body mass-index of greater than about 30.

### Example 1: Generation of Mono-Component and Bi-Component Liver Spheroids

Mono-component and bi-component liver spheroids were generated according to the protocol detailed in Example 1.1.

1.1 Generation of Healthy Mono-Component and Bi-Component Liver Spheroids from Human Primary Cells

### 1.1.1 Expansion and Sub-Culturing of Human Hepatic Stellate Cells (HSC)

For the purpose of the present Example, primary human hepatocytes (PHHs) or primary hepatic stellate cells (HSCs) derived from native liver tissue acquired from commercial manufacturers (HSC from ScienCell Research Laboratories (Cat #5300) and spheroid qualified PHH from Gibco (Cat no: HMCPSQ) or Lonza (Cat #HUCPI) after being screened for non-desired characteristics or qualities. For example, liver tissue donors are selected from between the ages of about 5 to about 40, screened for and excluded if history of alcohol usage and/or abnormally elevated levels of aspartate aminotransferase (AST) and/or alanine transaminase (ALT) and a body mass index greater than about 30. Cryopreserved HSCs were freeze-thawed according to the manufacturer's instruction (ScienCell, Catalog #5300). Briefly, the frozen vial from the liquid nitrogen was thawed in a 37° C. water bath with gentle rotation to ensure all the content was thawed. The thawed cells were emptied into 12 mL of freshly prepared hepatic stellate cell media (ScienCell, Catalog #5301) and gently mixed to prepare a homogeneous cell suspension. The cell suspension was pipetted to a T75 flask coated with poly-L-lysine (2 μg/cm2) and placed undisturbed in a 37° C. CO₂ incubator for at least 16 hours after which fresh hepatic stellate cell media was added to the culture flask. Once the cell culture reached 70% confluency, the stellate cell media was replaced every other day until the cell culture reached approximately 90% confluency. For sub-culturing, once the cell culture reached approximately 70% confluency, the stellate cell media was removed. The cell culture was washed once with DPBS. 2-3 mL 0.025% of Trypsin/EDTA solution was added to the T75 flask and incubated in 37° C. CO₂ incubator for 2-3 min. The T75 flask was observed under the microscope ensure dissociation of cells from the T75 flask. The T75 flask was gently tapped to detach any loosely bound cells from the T75 flask. 20-25 mL of complete media was added to the T75 flask for neutralizing the trypsin, the cell suspension was quickly transferred to a 50 mL centrifuge tube and centrifuged at 1000 rpm for 5 minutes. The supernatant was discarded, and the cells were re-suspended in fresh hepatic stellate cell media and a cell count was performed. For sub-culturing, the cell density of 5000 cells/cm2 was used.

### 1.1.2 Expansion and Sub-Culturing of Primary Human Hepatocytes (PHH)

Cryopreserved spheroid qualified PHHs were freeze-thawed at the time of seeding of cells for spheroids, according to the manufacturer's instruction. The cells were freeze-thawed according to the manufacturer's instruction. Cryopreserved vial was thawed in a 37° C. water bath with gentle rotation to ensure all the content was thawed. The thawed cells were transferred into 50 mL of hepatocyte thaw medium (Lonza: Catalog #: MCHT50 or Gibco: CM7500) and gently pipetted to achieve a homogeneous cell suspension. The cell suspension was centrifuged at ˜1000 rpm for 10 minutes and the supernatant was discarded. The cells were re-suspended in 5 mL of hepatocyte plating media (Media composition and preparation is listed in the section: Hepatocyte plating media preparation) and cell count was performed.

### 1.1.3 Generation of Mono-Component Liver Spheroids

Cell density of 2000 PHH per well in 96 well plate was calculated for the total number 96-well, Ultra-low attachment plates (Corning, USA) and the cells were diluted in plating media accordingly. 100 μL of cell suspension was pipetted to each well using a multi-channel pipette and the plate for centrifuged at 200×g for 3 min. The plate was kept in a 37° C., 5% CO₂ incubator undisturbed for 5 days. On Day 5, PHH plating media supplemented with FBS is replaced with PHH maintenance media (Thermo Cat: A15564). Day 5 onwards, the media was carefully replaced with fresh PHH maintenance media every 48 hours (half media change is done for media change for every 48 hours). Post Day 5, the plates were placed on an orbital shaker at a speed of less than 100 rpm to facilitate oxygen and nutrient exchange throughout the experiment. Generally, each well within the 96 well plate would consist of one spheroid, although it can be appreciated that a few well may consist of two spheroids that fuse together to form one spheroid during culture.

### 1.1.4 Generation of Bi-Component Liver Spheroids

Cell density of 2000 cells/well containing a 70:30 cell count ratio of PHH and HSC respectively (˜1400 PHHs and ˜600 HSC) were mixed in bi-component media described in Example 1.1.6.4 containing 1:1 of Hepatocyte plating media (Gibco) and HSC complete media. 100 μL cell suspension was pipetted to each well using a multi-channel pipette in a 96-well Ultra-low attachment plates (Corning, USA). The plate was kept in a 37° C., 5% CO₂ incubator undisturbed for 5 days. Once seeded, the plates were centrifuged at 200×g for 3 min and placed undisturbed in a CO₂ incubator for 5 days. On day 5, the media was carefully removed, and a 1:1 ratio of Hepatocyte maintenance media (Gibco, Cat: A15564) and Stellate cell culture media supplemented with the bullet kit components (HHSteC, ScienCell Cat No. 5301) was added. The plates were placed on an orbital shaker at a speed of less than 100 rpm to facilitate oxygen and nutrient exchange throughout the experiment. Generally, each well within the 96 well plate would consist of one spheroid, although it can be appreciated that a few well may consist of two spheroids that fuse together to form one spheroid during culture.

### 1.1.5 Maturation of Spheroids and Quality Check

After 5 days of culture in plating media, the plating media was carefully removed without disturbing the spheroids and replenished with 100 μL of hepatocyte maintenance for mono-component spheroids. For bi-component spheroids, the plating media was carefully removed without disturbing the spheroids and plenished with 100 μL of bi-component media (1:1 ratio of hepatocyte maintenance media and HSC media). The plates were put on orbital shaker with the speed of <100 rpm to facilitate gas and nutrient exchange and to prevent hypoxia in the core of the spheroids. The spheroids were placed in shaking conditions during disease induction and drug screening. The spheroids were allowed to mature for 48 h in the maintenance media. At day 7 post plating, the spheroids were checked for morphology (sphericity and compactness) by phase-contrast images obtained by microscopy to estimate spheroid diameter. Optimal diameter of the spheroids was found to be approximately 200-350 μm for the mono-component spheroids and approximately 300-450 μm for the bi-component spheroids.

Standard quality control studies such as viability immunofluorescence staining (Hoechst staining for nuclear localization, Ethidium homodimer staining for dead cells, and Calcein AM staining for live cells) and ATP assays were performed. Spheroids between days 5-7 post plating were used for further studies including induction of early-stage fibrosis, late-stage fibrosis, and NASH.

### 1.1.6 Cell Culture MediaPNDM-012/01US 345086-2113

### 1.1.6.1 Hepatocyte Plating Media

Hepatocyte plating media (Gibco; Catalog #: CM3000) contained 500 mL of William's E-media with 10% Fetal Bovine Serum, 1 μM Dexamethasone in DMSO, 1% Penicillin/Streptomycin (10,000 U/mL/(10,000 μg/mL), 4 μg/ml of Human Recombinant Insulin, 2 mM of GlutaMAX™ (200 mM/100 X), and 15 mM of HEPES, pH 7.4.

### 1.1.6.2 Hepatocyte Maintenance Media

Hepatocyte maintenance media (Gibco; Catalog #: CM4000) contained 500 mL of William's E-media with 10 mM of Dexamethasone in DMSO.

### 1.1.6.3 Cell Maintenance Cocktail-B

Cell Maintenance Cocktail-B contained 0.5% Penicillin/Streptomycin (10,000 U/mL/(10,000 μg/mL), ITS+ comprising: 6.25 μg/ml of human recombinant insulin, 6.25 μg/ml of human transferrin, 6.25 ng/ml of selenous acid, 1.25 mg/ml of bovine serum albumin, 5.35 μg/mL of linoleic acid, 2 mM of GlutaMAX™ (200 mM/100×), and 15 mM of HEPES, pH 7.4.

### 1.1.6.4 Bi-Component Plating Media and Maintenance Media

Bi-component plating media and maintenance media comprised 50 mL of primary hepatocyte plating media and 50 mL of hepatic stellate cells complete media (Gibco).

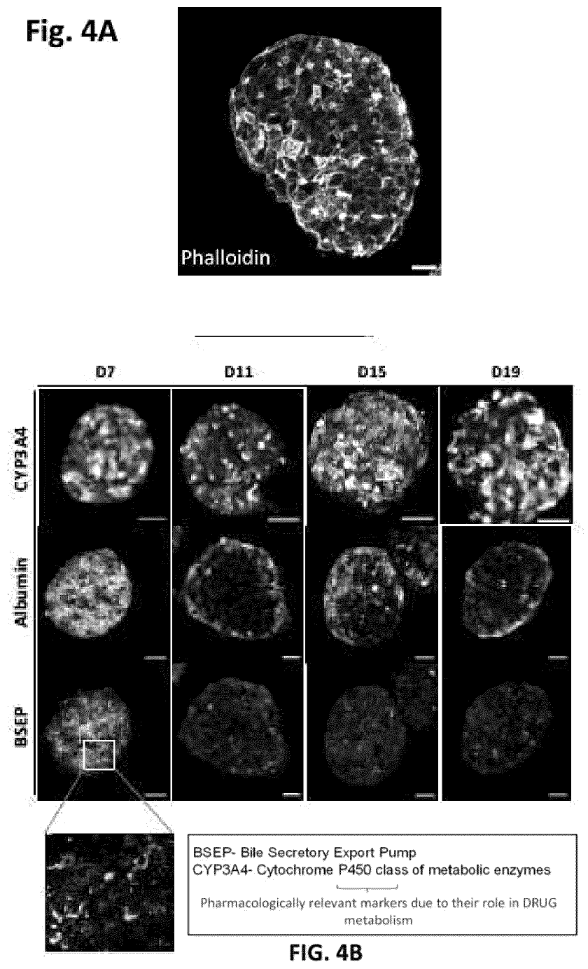

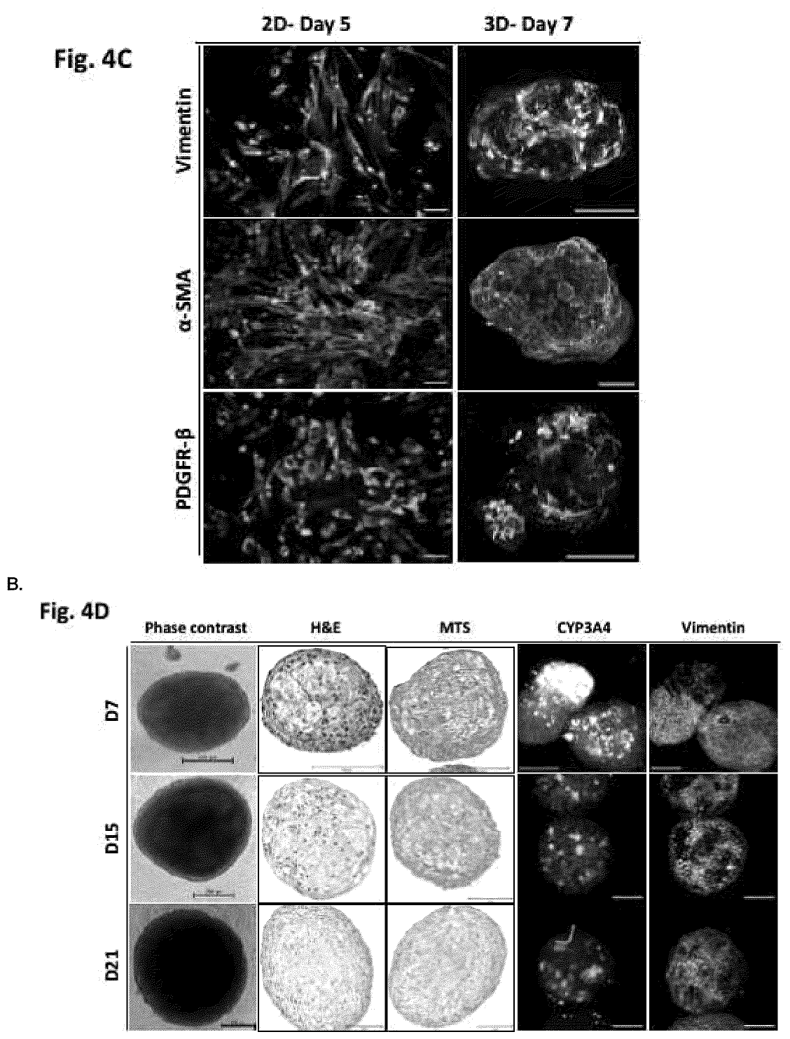

Mono-component liver spheroids were generated as described in Example 1. To determine if the liver spheroids generated demonstrate liver architecture and cell-cell interactions, liver spheroids were stained with fluorescent antibodies to actin and the nucleus. FIG. 4A illustrates a representative confocal microscopy image of a liver spheroid stained for phalloidin. As demonstrated in FIG. 4A, the liver spheroids generated in Example 1 show micro-architecture and cell-cell boundaries similar to native liver tissue.

Mono-component liver spheroids were cultured for a number of days and tested for functional markers at specific time points. The functional markers include Albumin, CYP34A, a class of metabolic enzymes, and BSEP, bile secretory export pump. FIG. 4B depicts representative microscopy images of mono-component liver spheroids at day 7, day 11, day 15, and day 19 immuno-stained with the appropriate antibodies that bind to the functional markers (CYP3A4, Albumin and BSEP). Mono-component liver spheroids were able to express the functional markers even until day 19.

To demonstrate the superiority of liver spheroids over two-dimensional cell culture, PHHs and HSCs were co-cultured according to standard techniques in 2D cell cultures and according to the bi-component liver spheroid protocol as demonstrated in Example 1. After 5 days, the co-culture of the PHHs and HSCs were immuno-stained for hepatocyte and hepatic stellate cell markers and imaged using confocal immunofluorescence microscopy. After 7 days, the bi-component liver spheroids were stained for hepatocyte and hepatic stellate cell markers and imaged using confocal immunofluorescence microscopy. FIG. 4C depicts representative images of a 2D cell culture of PHHs co-cultured with HSCs after 5 days in culture immuno-stained with Vimentin, α-SMA, and PDGFR-β, each of which are functional markers of healthy liver tissue. FIG. 4C also depicts representative images of liver spheroids composed of PHHs and HSCs after 7 days in culture, stained with Vimentin, α-SMA, and PDGFR-β. Although the 2D cell culture of PHHs co-cultured with HSCs had some expression of hepatocyte and hepatic stellate cell markers, the 2D cell culture lacked the structure and organization of native liver tissue. The liver spheroids had the structure of native liver tissue and expressed the hepatocyte and hepatic stellate cell markers. To further test liver spheroids for native liver tissue structure and function, liver spheroids were cultured for up to 21 days. At specific time points, liver spheroids were imaged or stained and imaged for morphology, microarchitecture, and functional hepatocyte and hepatic stellate cell markers. FIG. 4D depicts representative microscopy images of bi-component liver spheroids at day 7, day 15 or day 21. Representative images include phase-contrast, Hematoxylin and Eosin (H&E), MTS, and immunofluorescence staining for CYP3A4 and Vimentin. As demonstrated by the phase-contrast images, bi-component liver spheroids were able to maintain liver tissue structure and morphology up to 21 days in culture. The H&E and MTS staining demonstrate the liver spheroid's ability to express correct microarchitecture and collagen deposition even after 21 days in culture. CYP3A4 is a functional hepatocyte marker and the bi-component liver spheroids generated as described in Example 1 functionally express CYP3A4 up to 21 days. Likewise, Vimentin is a functional hepatic stellate cell marker and the bi-component liver spheroids functionally express Vimentin up to 21 days.

With Example 1 and FIGS. 4A-4D demonstrating mono-component and bi-component liver spheroids accurately display native liver tissue architecture and maintain liver tissue morphology, microarchitecture, collagen deposition and functional markers up to 21 days, mono-component and bi-component liver spheroids generated as described in Example 1 were induced to various diseased states to determine if disease state induction accurately depicted known states of NAFLD including steatosis, steato-hepatitis, early-stage fibrosis and late-stage fibrosis.

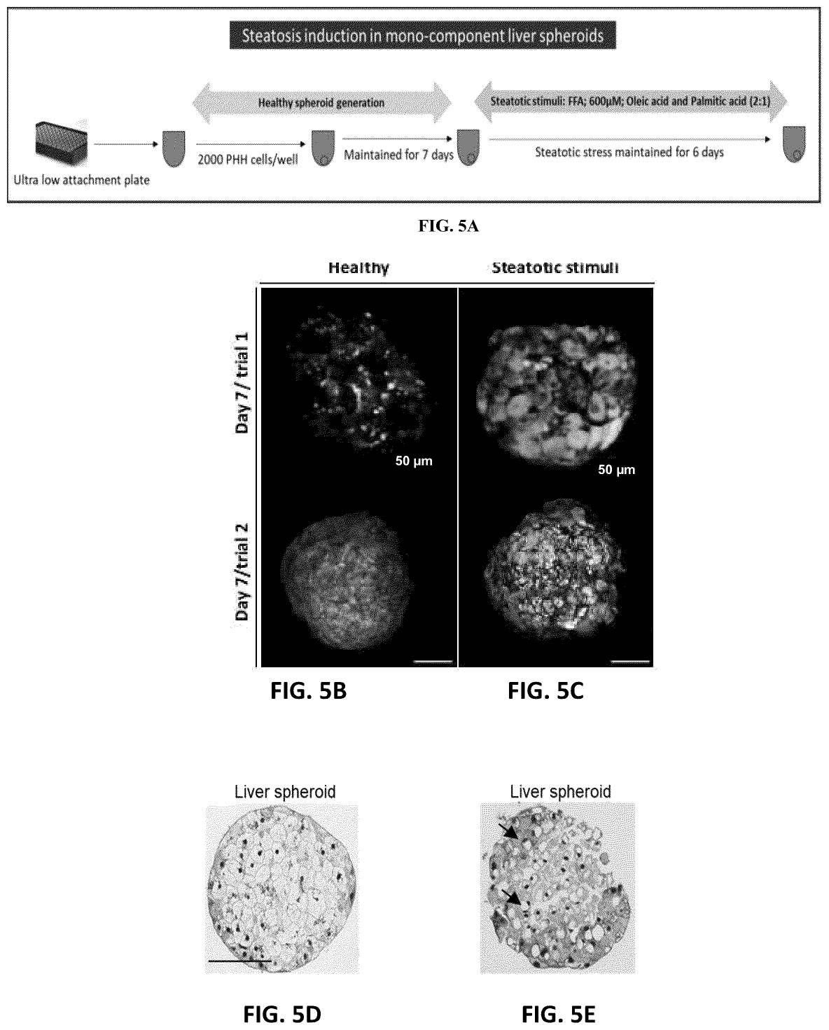

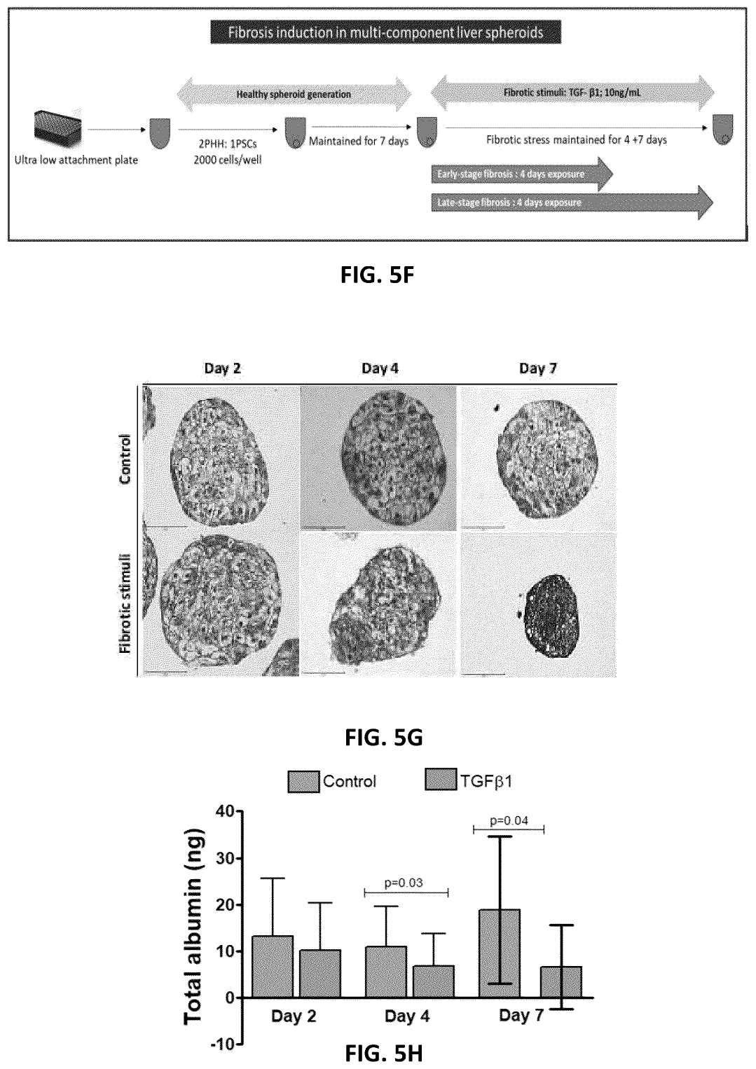

In inducing steatosis in liver spheroids, mono-component liver spheroids were subjected to steatotic stimuli, as illustrated in FIG. 5A. FIG. 5A depicts a schematic of a method of inducing a steatosis phenotype in mono-component spheroids.

### Example 2: A Steatosis Phenotype

Example 2 describes the protocol for inducing steatosis in mono-component liver spheroids.

### 2.1 Steatosis Induction in Healthy Mono-Component Liver Spheroids

Steatosis was induced in mono-component PHH liver spheroids using a cocktail of free-fatty acids (FFA) containing a 2:1 ratio of Oleic acid and Palmitic acid to the final concentration of 600 μM. After 7 days in culture, the media was removed from the mono-component liver spheroids without disturbing the mono-component liver spheroids ad replaced with steatotic media (media with the FFA cocktail) for 7 days with replenishment of the media occurring every alternative day. After 7 days of FFA treatment, the mono-component liver spheroids and the conditioned media were harvested from the 96 wells for further analysis.

A key phenotypic marker of steatosis in native liver tissue is the accumulation of intracellular lipids within the liver tissue. To confirm that a steatosis phenotype was induced in mono-component liver spheroids, steatosis induced liver spheroids and non-disease-induced “healthy” liver spheroids were stained with Nile Red and imaged. FIGS. 5B-5C depicts representative images of healthy (FIG. 5B) and steatosis induced (FIG. 5C) hepatocytes with accumulated intracellular lipids stained with Nile Red. Non-diseased liver spheroids demonstrated some lipid accumulation which is expected in healthy liver tissue, as depicted in FIG. 5B. However, as depicted in FIG. 5C, steatosis induced liver spheroids demonstrated an abundance of lipid accumulation compared to the healthy liver spheroids. Furthermore, healthy and steatosis induced liver spheroids were subjected to H&E staining and subsequently imaged.

FIGS. 5D-5E depicts representative microscopy images of H&E staining of a healthy liver spheroid and a steatosis induced liver spheroid. The steatosis induced liver spheroid (FIG. 5E) demonstrated an intracellular accumulation of lipid cells and a loss of spheroid structure compared to the healthy liver spheroid (FIG. 5D).

### Example 3: Induction of Fibrosis

The accumulation of extracellular matrix proteins including collagen within native liver tissue can lead to fibrosis. To induce fibrosis in liver spheroids, liver spheroids were subjected to pro-fibrotic stimuli that lead to the accumulation of extracellular matrix fibers such as collagen. FIG. 5F illustrates a schematic of a method of inducing a fibrotic phenotype in bi-component liver spheroids.

### 3.1 Fibrosis Induction in Bi-Component Liver Spheroids

Fibrosis was induced in bi-component liver spheroids produced in accordance with Example 1.1.4 by treatment with 10 ng/ml of TGF-β1. After 7 days in culture post-seeding, the media was removed from the bi-component liver spheroids and was replaced with the fibrosis inducing media containing TGF-β1. To induce early-stage fibrosis, the bi-component liver spheroids were cultured with the fibrosis inducing media for 4 days. To induce late-stage fibrosis, bi-component liver spheroids were cultured with the fibrosis inducing media for 7 days while replenishing fibrosis inducing media every alternative day. After either 4 days with the fibrosis inducing media (for early-stage fibrosis) or 7 days in with the fibrosis inducing media (for late-stage fibrosis), the liver spheroids and media were harvested from the 96 wells for further analysis.

FIG. 5G depicts representative microscopic images of Masson's Trichrome staining of healthy bi-component liver spheroids or fibrosis induced bi-component liver spheroids after 2, 4, and 7 days of induction of fibrosis. Masson's trichrome staining stains for collagen deposition and thus liver spheroids that have a fibrotic phenotype and have an abundance of collagen will stain positive for Masson's trichrome staining. Healthy liver spheroids after 2 days, 4 days, and 7 days in culture have relatively low expression of collagen. However, fibrotic induced bi-component liver spheroids show a mild accumulation for collagen after 4 days of fibrosis induction but show robust accumulation of collagen after 7 days of fibrosis induction. This indicates that 4 days of fibrotic induction in bi-component liver spheroids induces a mild fibrotic phenotype while 7 days of fibrotic induction demonstrates a robust fibrosis phenotype.

### Albumin and Fibronectin:

Albumin is a protein made and excreted by the liver into the blood stream. Low levels of secreted albumin correspond with a diseased liver. The supernatant from healthy bi-component liver spheroids and fibrotic induced bi-component liver spheroids was collected after 2 days, 4 days, and 7 days in culture and secreted albumin was detected by ELISA as per the manufacturer's protocol. FIG. 5H illustrates a graph depicting the quantification of secreted albumin of bi-component liver spheroids after 2, 4, and 7 days of fibrotic stimuli. There was a significant reduction in secreted albumin after 4 days and 7 days of fibrotic stimuli. Induction of early-stage fibrosis with 4 days of fibrotic stimuli and induction of late-stage fibrosis with 7 days of fibrotic stimuli lead to the accumulation of collagen within the diseased liver spheroids and a significant reduction in secreted albumin, similar to human fibrotic liver tissue.

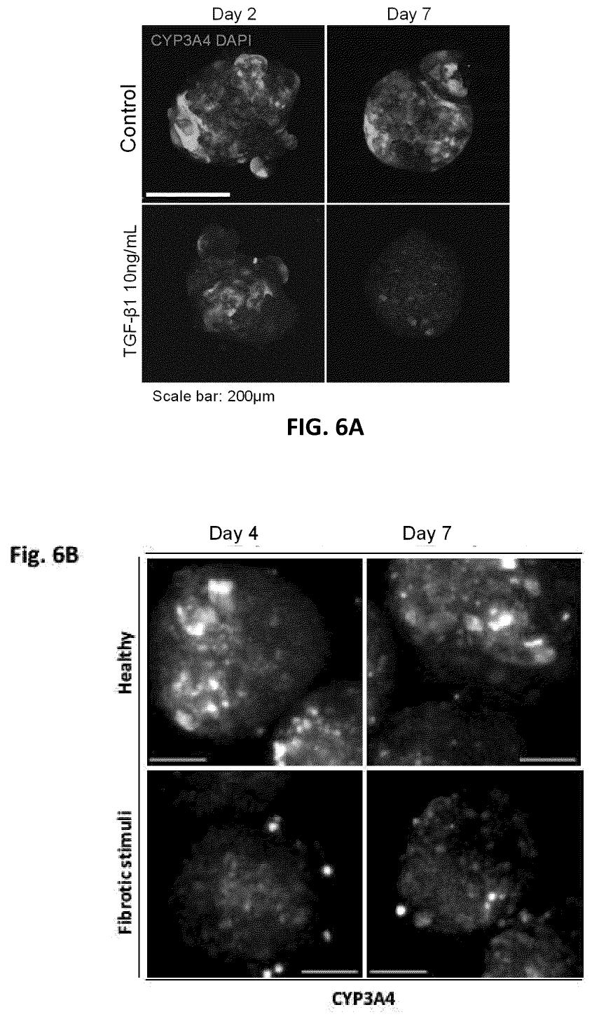

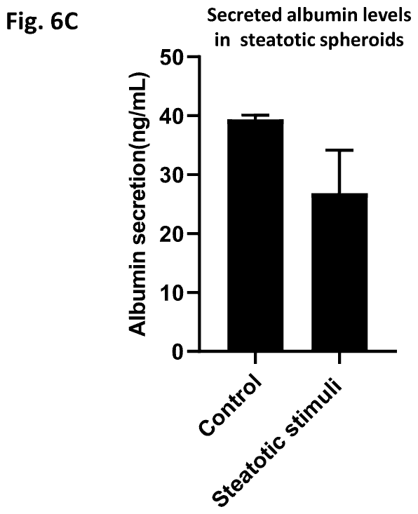

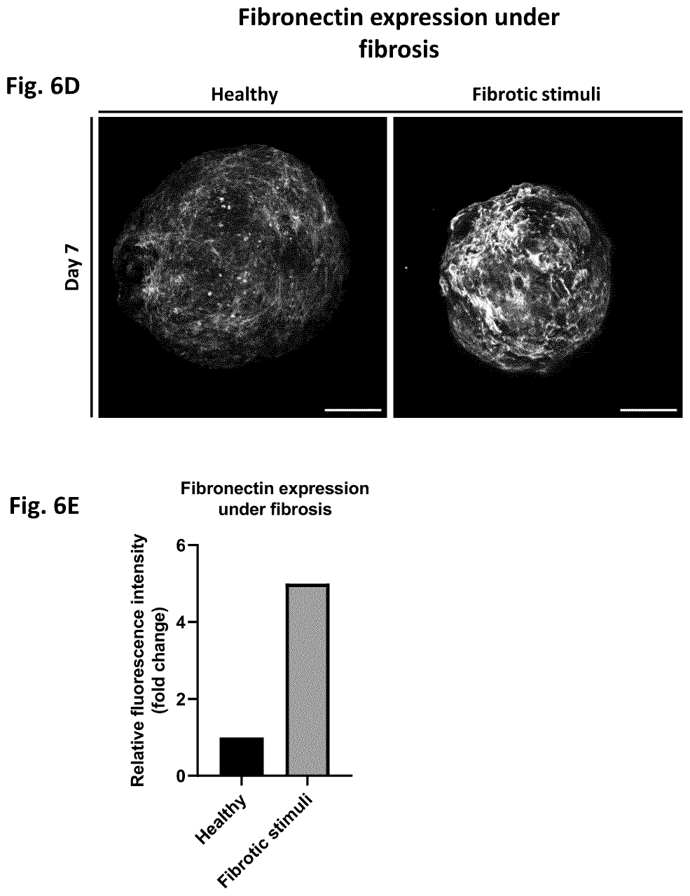

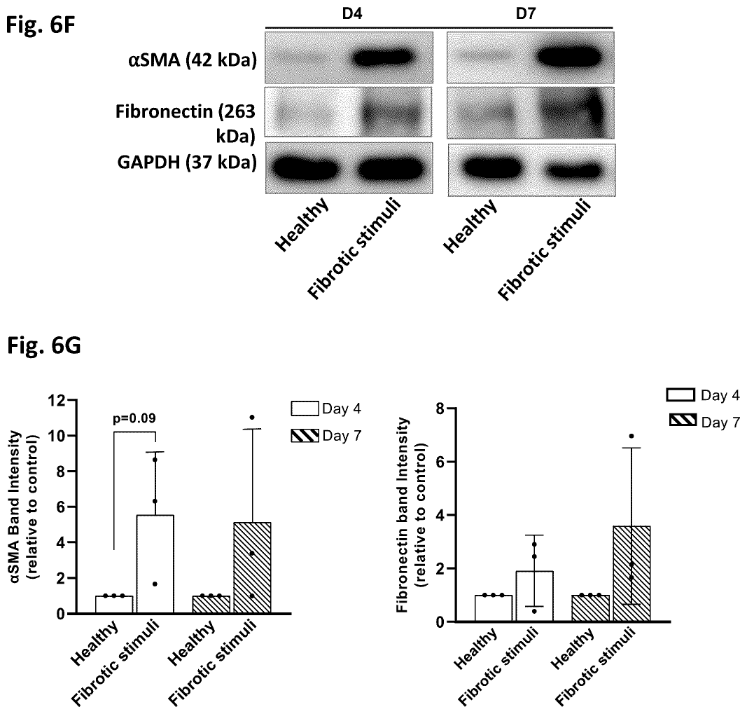

FIG. 6C is a bar graph showing the secreted albumin levels assessed via ELISA on day 7 of FFA treatment in PHH spheroids vs control. (n=3, *p<0.05, ns not significant).

Similarly, expression of fibronectin was also checked in multicomponent liver spheroid, and the data has been depicted in FIGS. 6D and 6E. FIGS. 6D and 6E show immunofluorescence micrographs and bar graph of quantified fluorescence intensity indicating variance in fibronectin expression in fibrotic multi-component liver spheroids (scale bar 100 μm). Marked increase in level of fibronectin can be observed in spheroids treated with fibrotic stimuli as compared to non-treated healthy spheroid.

### αSMA:

The levels of αSMA were tested after inducing fibrosis using fibrotic stimuli. The results are depicted in FIGS. 6F and 6G.

FIG. 6F shows micrographs obtained after SDS PAGE western blot assay showing the protein bands for α-SMA (band observed at 42 kDA) and fibronectin (band observed at 263 kDa) after 4 and 7 days TGF-β1 treatment in PHH and HSC spheroids (multi-component) vs control. GAPDH (band observed at 37 kDa) as loading control.

FIG. 6G depicts Bar graphs indicating quantified fold change in α-SMA and fibronectin expression (normalised to control) via densitometry analysis of western blotting assay micrographs in multicomponent liver spheroid. (n=3).

A significant increase can be observed in the level of αSMA in liver spheroids upon induction with fibrotic inducer.

FIGS. 6A-6B depict representative microscopy images of immunofluorescence staining of CYP3A4 in liver spheroids generated with hepatocytes from a first donor and a second donor after treatment with TGF-β1. To demonstrate that donor hepatocytes display consistent healthy or diseased phenotypes, bi-component liver spheroids were generated from hepatocytes from either a first donor or a second donor while the hepatic stellate cells were generated from the same hepatic stellate cell lots according to Example 1.1.3. Control liver spheroids were stained for CYP3A4 and DAPI after day 2 and day 7. Liver spheroids were subjected to either 2 days or 7 days of treatment with TGF-β1 (10 ng/mL) then stained for CYP3A4 and DAPI and imaged. As depicted in both FIGS. 6A and 6B, control liver spheroids demonstrated robust staining of CYP3A4, indicating liver spheroids still demonstrated normal liver cell function. However, after just 2 days of treatment with TGF-β1, liver spheroids showed reduced staining of CYP3A4 and after 7 days of treatment, most of the staining for CYP3A4 was gone, indicating after 2 days of TGF-β1, liver cell function was partially disrupted, and after 7 days of TGF-β1, liver cell function was severely disrupted.

### Example 4: Induction of NASH

To induce a NASH phenotype in liver spheroids, a steatosis phenotype was induced in liver spheroids followed by induction of fibrotic phenotype in liver spheroids.

### 4.1 Combinatorial Induction of Steato-Hepatitis in Bi-Component Liver Spheroids

For combinatorial induction of steatosis and fibrosis in bi-component liver spheroids, sequential treatment of FFA and TGF-β1 on the bi-component liver spheroids was performed. On day 7 post seeding, the healthy spheroids were treated with a FFA cocktail containing a 2:1 ratio of Oleic acid and Palmitic acid at a total concentration of 600 μM for 6 days with media change every other day. On day 6 post-treatment with FFA (at day 13 post-seeding), TGF-β1 (10 ng/ml) was added to the FFA containing media to initiate the combinatorial treatment of FFA and TGF-β1. On day 8 of steato-fibrosis induction (day 15 post-seeding), fresh media containing only TGF-β1 (10 ng/mL) was replenished and treated for another 2 days to accelerate the fibrosis induction. On day 10 post-disease induction (day 17 post-seeding) the spheroids and the conditioned media were harvested for the downstream characterization.

### Example 5: Transcriptome Analysis

While disease induction (steatosis, NASH, early-stage fibrosis, and late-stage fibrosis) in liver spheroids demonstrated accurate phenotypes similar to disease in humans, to determine if disease also demonstrated accurate gene expression changes within the liver spheroids, a bioinformatics approach was taken. While the standard readouts give the status of the selected inflammatory and fibrotic markers, transcriptomics captures the overall gene expression changes due to disease induction. Furthermore, transcriptomics allow mapping of the closest NASH stage specific model that compares with the human NASH liver through transcriptomics and hierarchical clustering.

### 5.1 Microarray and Transcriptomic Analysis of Liver Spheroid Samples

Total RNA was isolated from the Diseased spheroid models & their respective Healthy counterparts (control). For early-stage fibrosis (4-day treatment of TGF-β1) induction of liver spheroids, the total RNA of liver spheroids was collected from 3 different independent rounds of experiments. For late-stage fibrosis (7-day TGF-β1) induction of liver spheroids, the total RNA of liver spheroids was collected from 4 different independent rounds of experiments. For NASH (FFA+TGF-β1) induction of liver spheroids, the total RNA of liver spheroids was collected from one round of experiments. RNA samples were further outsourced to commercial facilities for Microarray-based hybridization protocols (Agilent).

The raw count files (output from Microarray) were used for the downstream bioinformatics analysis. In short, we have followed “Limma” based well established analysis pipeline (www.bioconductor.org/packages/devel/bioc/vignettes/limma/i nst/doc/usersguide.pdf) to determine Normalized Expression values (Transcriptome). The list of differentially expressed genes were used for plotting heatmaps, GO (Gene Ontology) enrichment analysis, and as input to Ingenuity Pathway analysis (IPA)-based Downstream analysis.

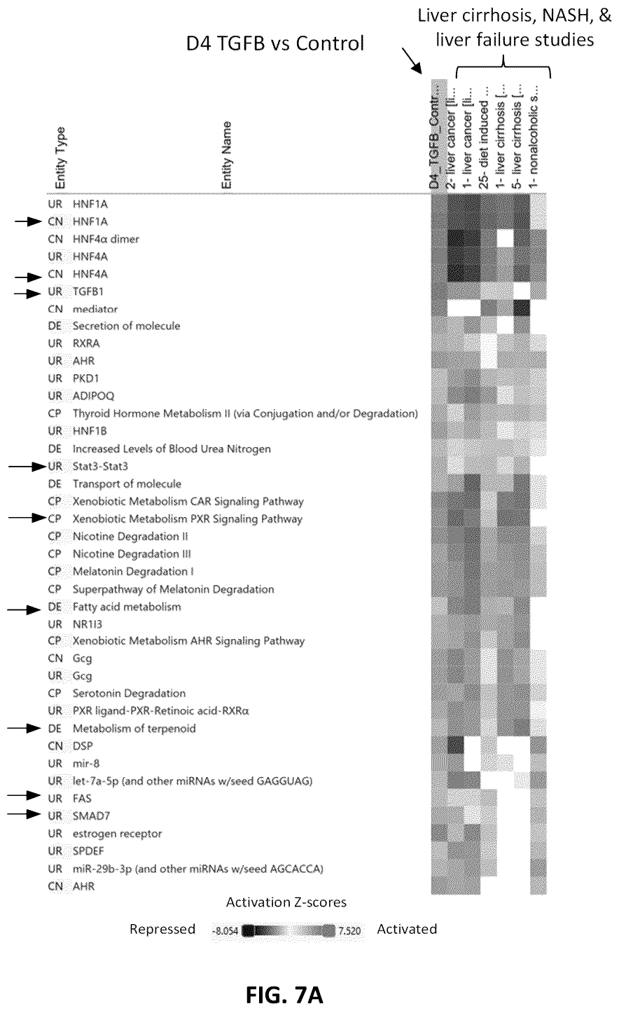

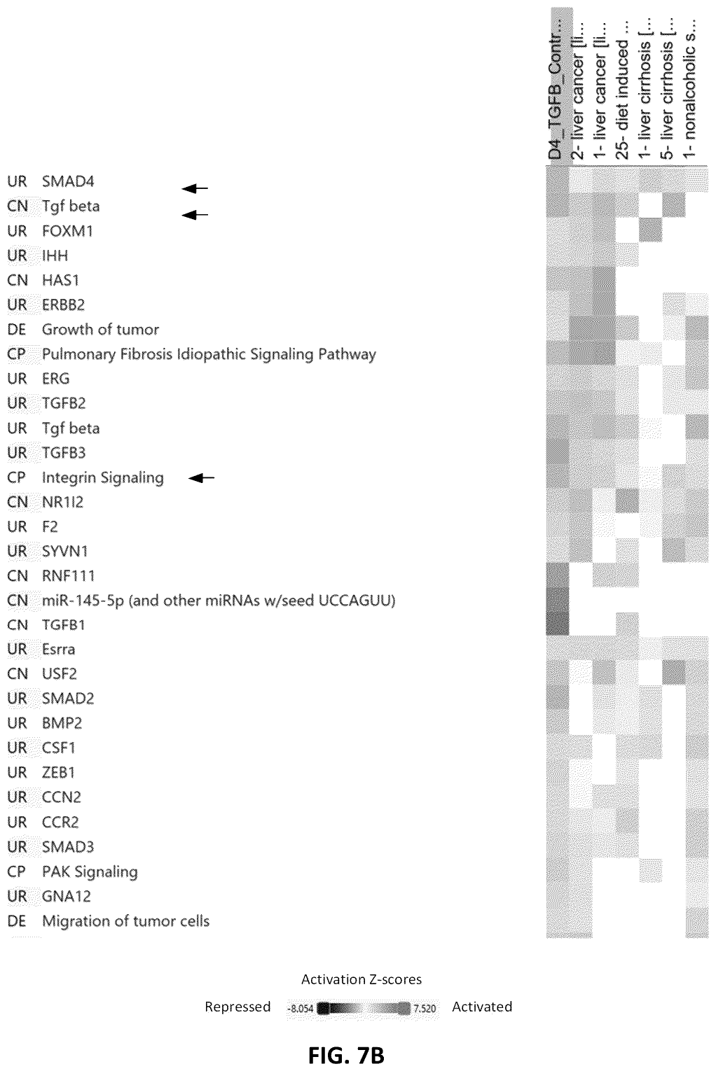

To determine if early-stage fibrosis induced liver spheroids approximate liver disease in human patients, a bioinformatics approach was taken. Early-stage fibrosis was induced in liver spheroids as described in Example 3.1 and total RNA was isolated for transcriptomic analysis as described in Example 5.1. The total RNA of healthy and early-stage fibrosis induced liver spheroids was compared to 6 published data sets having human gene expression data including liver cirrhosis, NASH, and liver failure data sets. FIGS. 7A and 7B depicts portions of a heat map of global gene expression in which each column represents a different liver or liver spheroid condition, and each row represents differential expression levels compared to respective controls of an individual gene or a GO category representing functional groups of multiple genes. The left-most column (D4_TGFB_Contr.) represents the gene expression profile of early-stage fibrosis induced liver spheroids. The remaining six columns to the right represent gene expression profiles based on published gene expression data related to liver cirrhosis, NASH, and liver failure. Overall, the gene expression profile of the early-stage fibrosis induced liver spheroids largely followed the published data sets from liver cirrhosis, NASH, and liver failure studies. Interestingly, in the early-stage fibrosis induced liver spheroids, the expression of HNF1A (hepatocyte nuclear factor-1 alpha), a key liver transcription factor, was strongly repressed as it was across the 6 published data sets. Likewise, HNF4A (hepatocyte nuclear factor-4 alpha), the most abundant DNA binding liver transcription factor, was also repressed in early-stage fibrosis induced liver spheroids as it was in the 6 published data sets. For example, the expression of genes in the PXR signaling and CAR signaling pathways (known to be altered in human NASH disease), STAT3 (known to be involved in fibrogenesis), and SMAD7 (a TGF-β1 antagonist) were repressed in the early-stage fibrosis induced liver spheroids.

Early-stage fibrosis induction in liver spheroids by 4 days of TGF-β1 treatment led to activated expression of some genes. As depicted in FIG. 7B, TGF-β expression was activated after TGF-β1 treatment. SMAD4, a transcription factor known to be pro-fibrotic in hepatic fibrosis, was also activated in early-stage fibrosis induced liver spheroids and the 6 published data sets. Integrin signaling was also activated in early-stage fibrosis induced liver spheroids and some of the 6 published data sets. As illustrated in FIGS. 7A-7B, inducing early-stage fibrosis in liver spheroids with 4 days of TGF-β1 treatment leads to similar gene expression profiles in the liver spheroids as published data sets of human gene expression in liver cirrhosis, NASH, and liver failure.

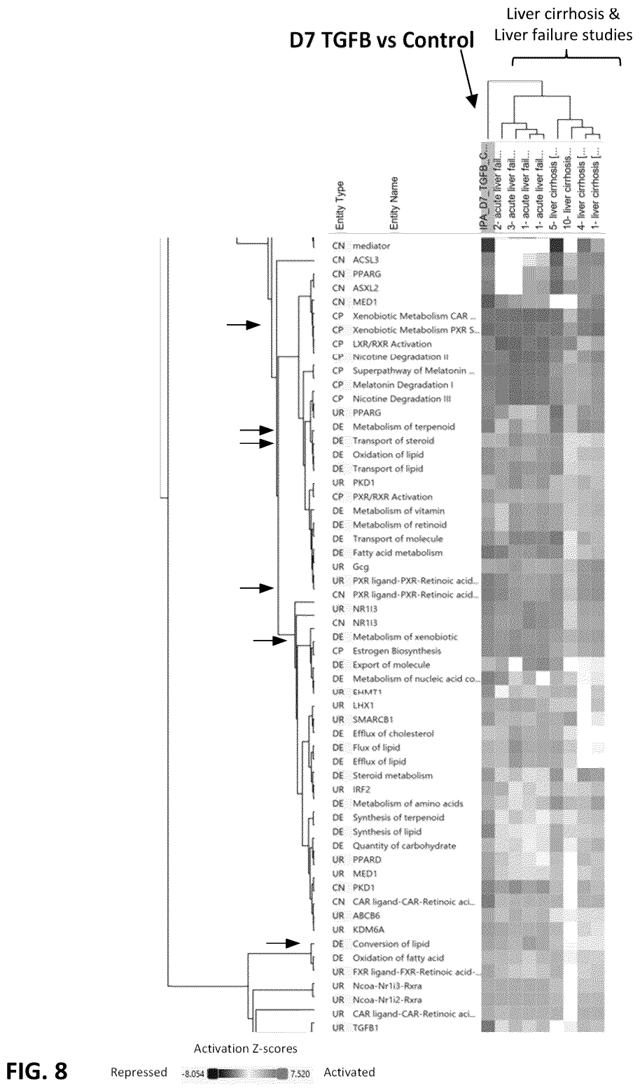

To determine if late-stage fibrosis induced liver spheroids approximate liver disease in human patients, a similar bioinformatics approach was taken. Late-stage fibrosis was induced in liver spheroids (7 days of TGF-β1 treatment) as described in Example 3.1 and total RNA was isolated for transcriptomic analysis as described in Example 5.1. The total RNA of healthy and late-stage fibrosis induced liver spheroids was compared to 6 published data sets having human gene expression data including liver cirrhosis, and liver failure data sets. FIG. 8 depicts a portion of a heat map in which each column represents a different liver or liver spheroid condition, and each row represents differential expression levels compared to respective controls of an individual gene or a GO category representing functional groups of multiple genes. The left-most column (IPA_D7_TGFB_C) represents the gene expression profile of late-stage fibrosis induced liver spheroids. The remaining eight columns to the right represent gene expression profiles based on published gene expression data related to liver cirrhosis and liver failure. Overall, gene expression of late-stage fibrosis induced liver spheroids followed the gene expression data from published liver cirrhosis and liver failure studies.

More specifically, induction of late-stage fibrosis in liver spheroids leads to repression of genes in key liver pathways including metabolism (xenobiotic, retinoid, fatty acid), conversion and transport of lipids, and oxidation of fatty acids similar to the gene expression data from the published liver cirrhosis and liver failure studies. Induction of late-stage fibrosis in liver spheroids by 7 days of TGF-β1 treatment led to an activation of the expression of TGF-β1. As illustrated in FIG. 8, inducing late-stage fibrosis in liver spheroids with 7 days of TGF-β1 treatment leads to similar gene expression profiles in the liver spheroids as published data sets of human gene expression in liver cirrhosis and liver failure, suggesting that induction of late-stage fibrosis in liver spheroids by 7 days of TGF-β1 treatment may be a viable and accurate alternative for studying late-stage fibrosis in vivo.

To examine NASH induction in liver spheroids, NASH was induced in liver spheroids as described in Example 4.1, total RNA was isolated for transcriptomic analysis as described in Example 5.1, and microarray-based assay was performed on the total RNA followed by a bioinformatic analysis of the gene expression data from the microarray-based assay.

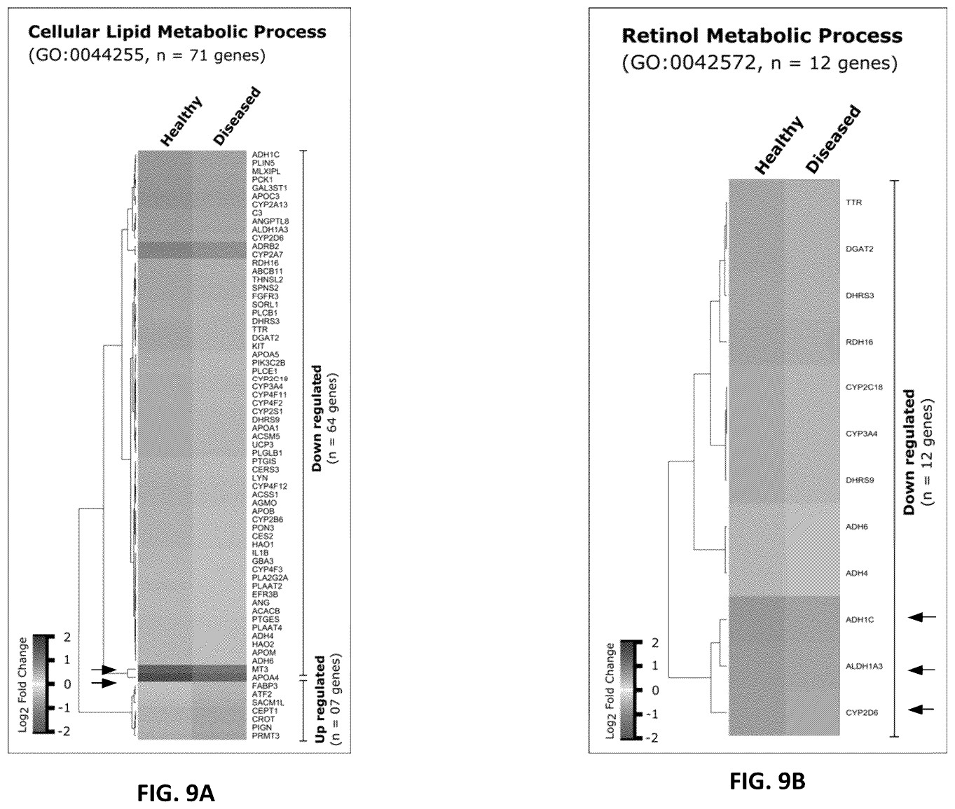

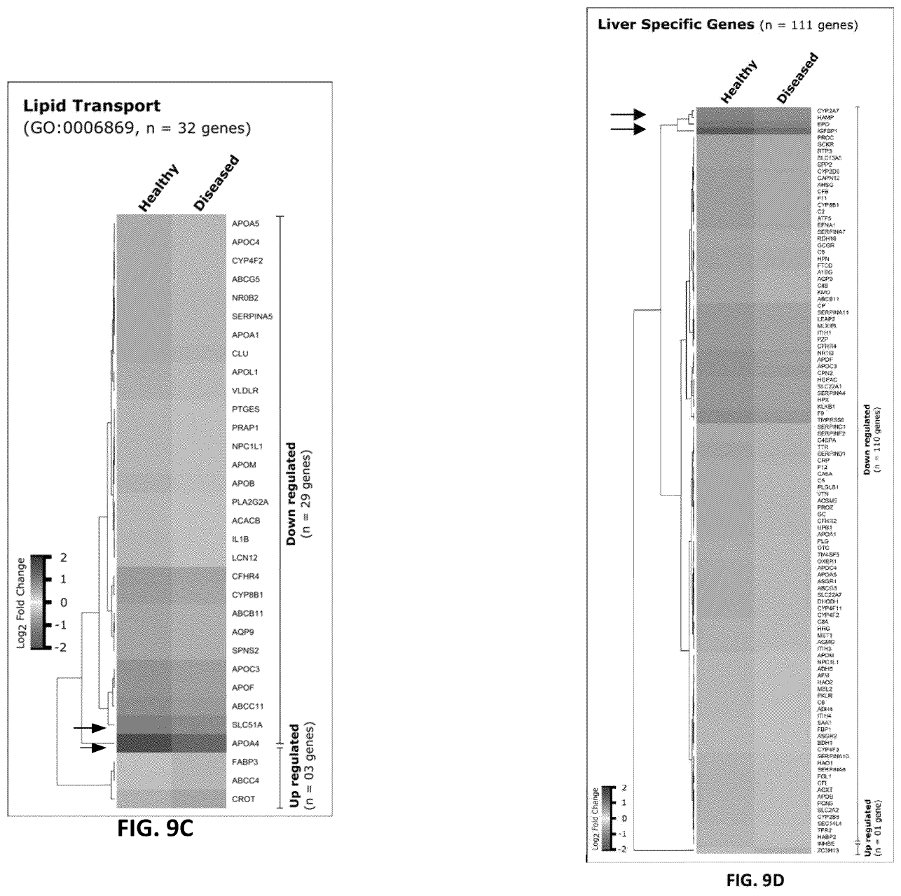

FIGS. 9A-9D depict heat maps of the gene expression of key processes in healthy liver spheroids (“Healthy”) compared to NASH induced liver spheroids (“Diseased”), based on the microarray data. As depicted in FIG. 9A, in evaluating 71 genes in the cellular lipid metabolic process, NASH induced liver spheroids demonstrated 64 genes that were downregulated compared to the healthy liver spheroids having those same 64 genes being upregulated. The 64 genes that were downregulated in NASH induced liver spheroids include several members of the CYP family, Apo A, Apo B, IL-β, along with many other genes. Specifically, two genes demonstrated a two-log fold reduction in expression as compared to healthy liver spheroids, MT3, a member of the metallothionein family of genes, and APOA4, an apolipoprotein. While 64 of the 71 genes were down regulated in the NASH induced liver spheroids, 7 genes were upregulated in the NASH induced liver spheroids with the same 7 genes being downregulated in healthy liver spheroids including Fatty Acid Binding protein 3 (FABP3), choline/ethanolamine phosphotransferase 1 (CEPT1), Carnitine O-Octanoyltransferase (CROT1), and others.

Retinol, more commonly known as Vitamin A, is important in many processes within the body. For example, Vitamin A is known activation or repression of many genes by binding to numerous nuclear transcription factors. FIG. 9B depicts a heat map of 12 genes within the retinol metabolic process. Interestingly, all 12 genes interrogated within the retinol metabolic process were downregulated compared to healthy controls. These genes include member of the alcohol dehydrogenase family, Aldehyde dehydrogenase family, and member of the CYP family. Specifically, ADH1C known as Alcohol Dehydrogenase IC, ALDH1A3 known as Aldehyde Dehydrogenase Family 1 Member A3, and CYP2D6, a member of the Cytochrome P450 Family 2 demonstrated at least a log fold change in gene expression compared to healthy liver spheroids. This suggests that NASH induction in liver spheroids by FFA treatment followed by TGB-β treatment significantly impairs liver spheroids by disrupting the retinol metabolism process.

Another significant process mediated by liver issue is lipid transport. FIG. 9C illustrates a heat map of 32 genes within the lipid transport process. Of those 32 genes, 29 genes were down regulated in NASH induced liver spheroids compared to control. 3 genes were upregulated in NASH induced liver spheroids compared to healthy liver spheroids. Specifically, SLC51A, an organic solute transporter involved in bile secretion, and APOA4, an apolipoprotein, demonstrated at least a two-log fold reduction in gene expression in NASH induced liver spheroids compared with healthy liver spheroids. Other genes such as APOF apolipoprotein F, ABCC11 ATP binding cassette subfamily C member 11, APOC3 apolipoprotein C3, and CYP8B1 cytochrome P450 family 8 subfamily B member 1 demonstrated at least a one log fold change in gene expression in NASH induced liver spheroids compared with healthy liver spheroids.

FIG. 9D depicts a heat map of 111 liver specific genes in NASH induced liver spheroids and healthy controls. Of the 111 liver specific genes, 110 were down regulated in NASH induced liver spheroids and I gene was upregulated. Specifically, IGFBP1, Insulin-Like Growth Factor-Binding Protein 1, and EPO, erythropoietin demonstrated a two-log fold reduction in gene expression in NASH induced liver spheroids compared to healthy controls. Out of the 111 liver specific genes, the 1 gene that was upregulated in NASH induced liver spheroids compared to healthy liver spheroids was ZC3H13, a Zinc Finger protein that enables RNA binding activity and is thought to be involved in mRNA methylation.

FIGS. 9A-9D demonstrate that NASH induction through free fatty acid treatment followed by TGF-β1 treatment leads to a significant reduction in gene expression within key pathways in the liver spheroids compared to healthy liver spheroids.

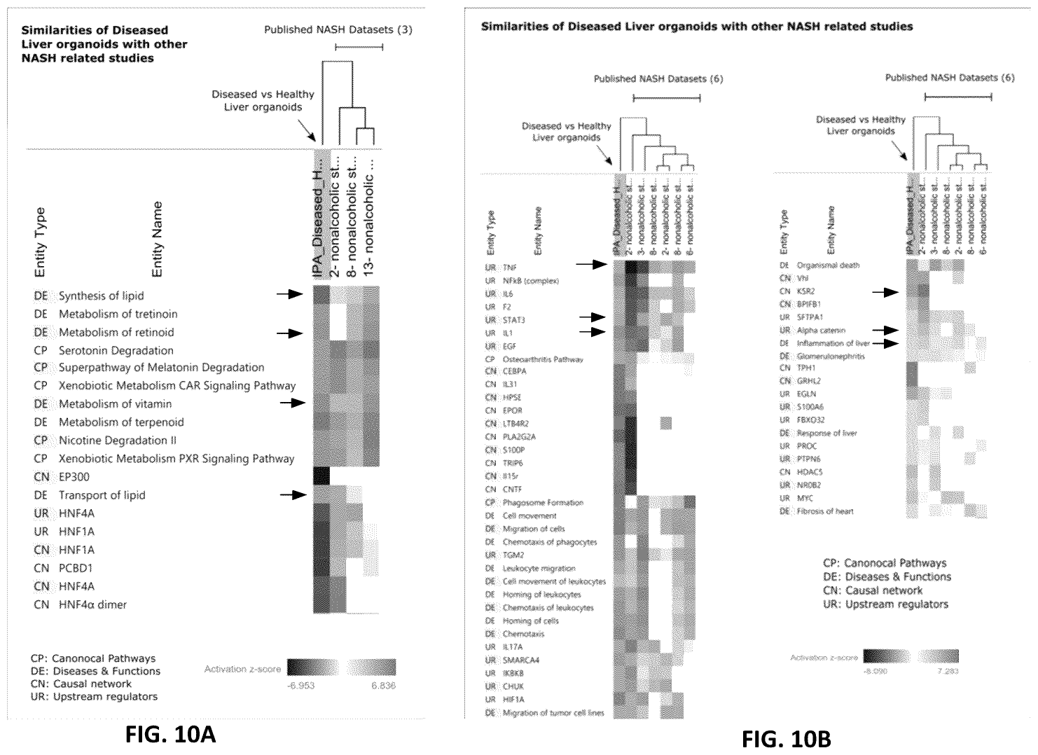

To show that NASH induction in liver spheroids demonstrates gene expression signatures that are similar to clinical human NASH samples, microarray-based gene expression data from NASH induced liver spheroids was compared with 3-6 separate published human NASH data sets. Each of FIGS. 10A-10B shows a heatmap in which each column represents a different liver or liver spheroid condition, and each row represents differential expression levels compared to respective controls of an individual gene or a GO category representing functional groups of multiple genes. FIG. 10A depicts a heat map of gene expression data from the NASH induced liver spheroids (left-most column, “IPA_Diseased_H . . . ”) and control liver spheroids compared with 3 published data sets from human NASH studies. Key pathways of the liver including key pathways responsible for a NASH phenotype (e.g., lipid synthesis, lipid transport, metabolism including metabolism of terpenoids and fatty acids, PXR signaling, inflammation, FAS, and metabolism signaling) demonstrated gene expression changed in NASH induced liver spheroids as it was in the 3 published data sets.

FIG. 10B depicts a heat map of gene expression data from NASH induced liver spheroids and control liver spheroids compared with 6 published data sets from human NASH studies. The left of FIG. 10B depicts the genes that were downregulated in both NASH induced liver spheroids and some of the 6 published data sets, including genes such as TNF, STAT3, and IL-1, key inflammatory cytokines, and transcription factors. The right of FIG. 10B depicts genes or pathways that were upregulated in both NASH induced liver spheroids and some of the 6 published data sets, including genes in the pathway related to the inflammation of the liver (including genes such as insulin like growth factor binding protein 1, integrin subunit beta 2, TNF superfamily member 10 or the like), alpha catenin a protein thought to regulate actin filament assembly, KSR2, a kinase suppressor of Ras 2, and genes related to fibrosis (including genes such as TNF alpha induced protein 3, adrenoceptor beta 2, matrix metallopeptidase 9, lipocalin 2, and iodothyronine deiodinase 3).

Through FIGS. 9A-9D and FIGS. 10A-10B, it was demonstrated that the induction of NASH through a combination treatment of free fatty acids and TGF-β1 in bi-component liver spheroids demonstrated gene expression changes that compared closely with human non-alcoholic steato-hepatitis through transcriptomics and hierarchical clustering. The sequential free fatty acid and TGF-β1 treatment in bi-component liver spheroids provide a robust NASH phenotype, while carrying over similar gene expression changes as seen in human NASH, indicating that NASH induced liver spheroids can be a model for studying NASH. Furthermore, not only were the gene expression changes within NASH induced liver spheroids similar to gene expression in human NASH samples, but also the gene expression changes were within canonical pathways responsible for the NASH phenotype including intracellular lipid accumulation, fibrosis, and inflammation. This indicates that the mechanistic aspect of NASH is also being modelled within NASH induced liver spheroids.

Gene expression data from early-stage fibrosis induced, late-stage fibrosis induced, and NASH induced liver spheroids was acquired as set out in Example 4. Table 1 compares Gene Accession Omnibus (GEO) accession number GSE160016 that provides expression patterns of genes that are differentially expressed in the liver of NAFLD patients compared to control samples, and the Gene Accession Omnibus (GEO) accession numbers GSE33814 and GSE105127 that provides expression patterns of genes that are differentially expressed in the liver of NASH patients compared to control samples with differential expression in NASH induced liver spheroids compared to non-induced control liver spheroids (“TGF-β1+FFA”), differential expression in late-stage fibrosis induced liver spheroids compared to non-induced control liver spheroids (“TGF-β1, D7”); and differential expression in early-stage fibrosis induced liver spheroids compared to non-induced control liver spheroids (“TGF-β1, D4”). Canonical pathways in both the clinical data sets and the disease induced liver spheroids were identified, compared, and the comparisons were scored with a z score. The z score represents the cumulative scores in percentages with respect to the overall canonical pathway-based similarity including the fate of those canonical pathways and the state of activation or repression of genes within the canonical pathways. For example, a z score of 100 indicates a complete match of the state of the canonical pathways including activation or repression between a clinical data set and a data set from a disease induced liver spheroid. A z score of 25 indicates a high degree of variance in the state of the canonical pathways including activation and/or repression between a clinical data set and a data set from a diseased induced liver spheroid.

TABLE 1
Scoring of transcriptomics-based similarities of disease induced spheroid models with published human clinical liver disease data sets

| | | | | | | Canonical Pathway based | | |
|---|---|---|---|---|---|---|---|---|
| | | | | | | similarity (z-scores) | | |
| | | | | | | Diseased | Diseased | Diseased |
| | | | | | | (TGF-β + | (TGF-β | (TGF-β |
| | | | | | | FFA | Treated, | Treated, |
| | Analysis Name | | | | | Treated) | Day 4) vs | Day 7) vs |
| | (Publicly | Diseased | Tissue | Samples stage | | vs | Control | Control |
| Row | available Data) | States | (Human) | details | Comparison_IDs | Control | (Day 4) | (Day 7) |
| 1 | nonalcoholic fatty | nonalcoholic | liver | NAFLD | GSE160016.GPL24676.DESeq2R.test1 | 70.71 | 59.16 | 59.16 |
| | liver disease | fatty liver | | (stages | | | | |
| | (NAFLD) [liver] | disease | | unknown) vs | | | | |
| | NA 6087 | (NAFLD) | | Normal tissue | | | | |
| 2 | nonalcoholic | nonalcoholic | liver | Fibrosis grade | GSE33814.GPL6884.test2 | 63.25 | 59.16 | 54.77 |
| | steatohepatitis | steatohepatitis | | (F3-F4) vs | | | | |
| | (NASH) [liver] | (NASH) | | Normal tissue | | | | |
| | NA 18691 | | | | | | | |
| 3 | nonalcoholic | nonalcoholic | liver | Disease State: | GSE105127.GPL16791.DESeq2R.test8 | 63.25 | 59.16 | 50.00 |
| | steatohepatitis | steatohepatitis | | Tissue | | | | |
| | (NASH) [liver] | (NASH) | | Region => | | | | |
| | NA 1637 | | | periportal −> | | | | |
| | | | | nonalcoholic | | | | |
| | | | | steatohepatitis | | | | |
| | | | | (NASH) vs | | | | |
| | | | | normal | | | | |
| | | | | control tissue | | | | |

Out of the three induced liver spheroid samples, the liver spheroids treated with the combination of steatosis inducers (FFA) and TGF-β1 demonstrated a high similarity in the states of canonical pathways compared to the clinical NAFLD sample (row 1), indicated by the 70.71 z score. Early-stage fibrosis induction and late-stage fibrosis induction in liver spheroids demonstrated some variance in the state of canonical pathways compared to the clinical NAFLD sample, as indicated by comparatively reduced z scores of 59.16 and 59.16, respectively. This indicates that the states of the canonical pathways in the disease induced liver spheroids are most similar to clinical NAFLD when the liver spheroids are induced with a NASH phenotype using steatosis inducers including FFA and inflammation including TGF-β1.

In comparing the data set from the clinical sample of NASH that included fibrosis graded F3-F4 (row 2) to the data sets from the disease induced liver spheroids, the NASH induced liver spheroids had the highest z score of 63.25 compared to slightly lower z scores of 59.16 for the early-stage fibrosis induced liver spheroids, and 54.77 for the late-stage fibrosis induced liver spheroids. This indicates that out of the disease induced liver spheroids, NASH induced liver spheroids demonstrated the highest similarity in the state of canonical pathways to the clinical sample of NASH.

Similarly, the comparison of the data set from the second clinical sample of NASH (row 3) to the data sets from the disease induced liver spheroids yielded similar results. The NASH induced liver spheroids demonstrated a similar trend as rows 1 and 2, having the highest z score among the disease induced liver spheroids. Early-stage induced fibrosis and late-stage induced fibrosis also followed the trend of the previous rows, having reduced z scores. This indicates that NASH induced liver spheroids demonstrated the least variance in the states of the canonical pathways in the comparison between the clinical data sets and the disease induced liver spheroid data sets.

Table 1 indicates that the gene expression within canonical pathways displayed in clinical NASH and NAFLD samples were most similar to the gene expression within canonical pathways in NASH induced liver spheroids compared to early-stage fibrosis induced, and late-stage fibrosis induced liver spheroids. Taking the data from Table 1 with the data described above in FIGS. 9A-10B, it can be concluded that the states of the canonical pathways in NASH induced liver spheroids are most similar to clinical samples including NASH and that the differential gene expression in the NASH induced liver spheroids demonstrate lipid accumulation including a reduction in genes related to lipid metabolism, fibrosis including a reduction in genes related to metabolism, and genes related to inflammation, indicating that NASH induction in liver spheroids also models the mechanistic aspect of NASH

To further show that the NASH induction protocol in liver spheroids demonstrates gene expression signatures that are similar to clinical human NASH samples, gene expression profiles were compared between the following samples:
1. microarray-based gene expression data from NASH induced liver spheroids, induced with the protocol described in Example 4.1 (sequential treatment with a FFA cocktail consisting of Oleic acid and Palmitic acid at a 2:1 weight ratio, followed by TGF-β1), with the microarray-based data acquired as described in Example 5.1.
2. Early-stage fibrosis induced liver spheroids, induced with the protocol described in Example 3.1 (4 day treatment with TGF-1), with the microarray-based data acquired as described in Example 5.1;
3. Early-stage fibrosis induced liver spheroids, induced with the protocol described in Example 3.1 (7 day treatment with TGF-β1), with the microarray-based data acquired acquired as described in Example 5.1; and
4. One or more published gene expression profiles of clinical liver disease states, based on liver samples taken from patients diagnosed with a liver disease (steatohepatitis. NAFLD, or steatosis)

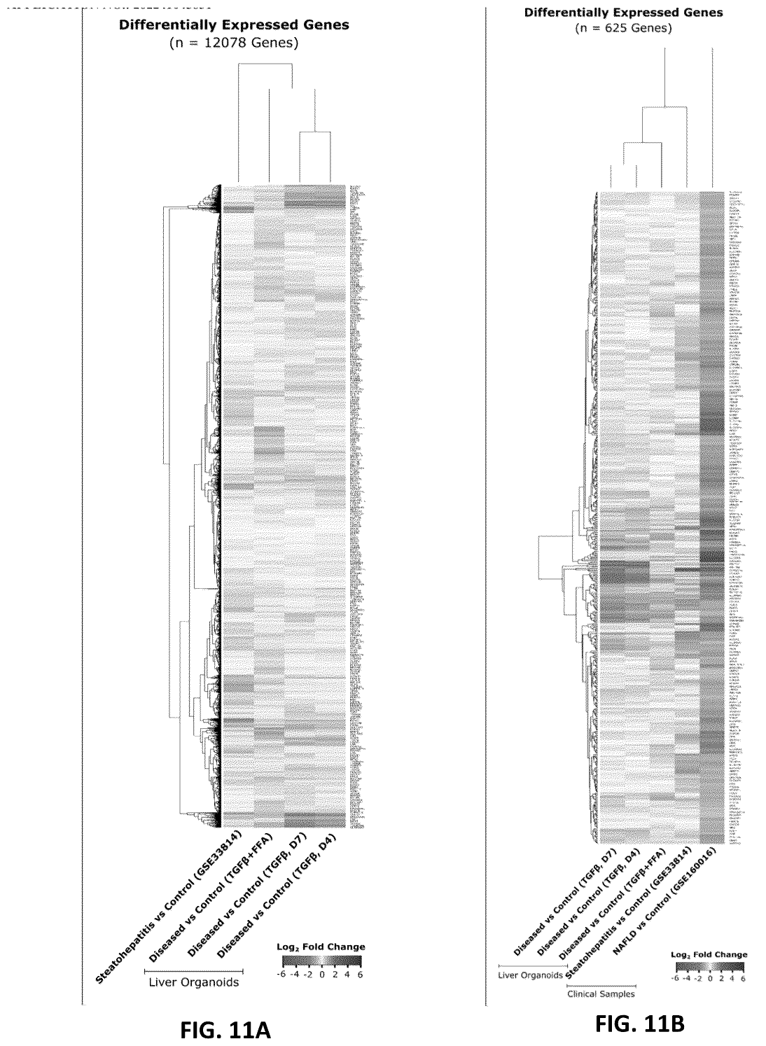

Each of FIGS. 11A-11B shows a heatmap in which each column represents a liver spheroid disease state induced in accordance with an embodiment of the disclosure, or a clinical liver disease state, and each row represents differential expression levels compared to respective controls of an individual gene or a GO category representing functional groups of multiple genes. The columns in each heatmap are arranged based on a hierarchical clustering analysis of the gene expression patterns in each of liver disease states (either a liver spheroid disease state or a clinical liver disease state). Pairs of liver disease states that cluster in the vicinity of each other with respect to their respective gene expression patterns are arranged as adjacent columns, and the brackets connecting the columns reflect the degree of similarity between the respective gene expression patterns of the different liver disease states.

FIG. 11A compares the following gene expression patterns:
(1) Gene Accession Omnibus (GEO) accession number GSE33814 that provides expression patterns of genes that are differentially expressed in the liver of steatohepatitis patients compared to steatosis patients' samples or control samples (“GSE33814”);
(2) Differential expression in NASH induced liver spheroids compared to non-induced control liver spheroids (“TGF-β1+FFA”);
(3) Differential expression in late-stage fibrosis induced liver spheroids compared to non-induced control liver spheroids (“TGF-β1, D7”); and
(4) Differential expression in early-stage fibrosis induced liver spheroids compared to non-induced control liver spheroids (“TGF-β1, D4”).

The heatmap is limited to the 12,078 genes that were designated to be differentially expressed compared to the respective controls in each of the following two sets: (1) the three liver spheroid disease states and (2) the GSE33814 data set. As shown in the bracketing pattern of the columns in FIG. 11A, out of the three liver spheroid disease states, the differential gene expression pattern of the NASH induced liver spheroids was determined to most closely resemble the differential gene expression pattern seen in the liver of steatohepatitis patients. FIG. 11B compares the following gene expression patterns:
(1) Differential expression in late-stage fibrosis induced liver spheroids compared to non-induced control liver spheroids (“TGF-β1, D7”); and
(2) Differential expression in early-stage fibrosis induced liver spheroids compared to non-induced control liver spheroids (“TGF-β1, D4”);
(3) Differential expression in NASH induced liver spheroids compared to non-induced control liver spheroids (“TGF-β1+FFA”);
(4) GEO accession number GSE33814 that provides expression patterns of genes that are differentially expressed in the liver of steatohepatitis patients compared to steatosis patients' samples or control samples (“GSE33814”); and
(5) GEO accession number GSE160016 that provides expression patterns of genes that are differentially expressed in the liver of NAFLD patients compared to control subjects.

The heatmap is limited to the 625 genes that were designated to be differentially expressed compared to the respective controls in each of the following three sets: (1) the three liver spheroid disease states, (2) the GSE33814 data set, and (3) the GSE16001 data set. As shown in the bracketing pattern of the columns in FIG. 11B, the differential gene expression pattern of the NASH induced liver spheroids was determined to more closely resemble the differential gene expression pattern seen in the liver of steatohepatitis patients (GSE33814) that the differential gene expression pattern seen in the liver of NALFD patients (GSE16001). In addition, out of the three liver spheroid disease states (NASH, early-stage fibrosis and late-stage fibrosis), the differential gene expression pattern of the NASH induced liver spheroids was determined to most closely resemble the differential gene expression pattern seen in the liver of steatohepatitis patients (GSE33814).

Another heatmap (not shown) compares the following gene expression patterns:
(1) Differential expression in late-stage fibrosis induced liver spheroids compared to non-induced control liver spheroids (“TGF-β1, D7”); and
(2) Differential expression in early-stage fibrosis induced liver spheroids compared to non-induced control liver spheroids (“TGF-β1, D4”);
(3) Differential expression in NASH induced liver spheroids compared to non-induced control liver spheroids (“TGF-β1+FFA”);
(4) GEO accession number GSE33814 that provides expression patterns of genes that are differentially expressed in the liver of steatohepatitis patients compared to steatosis patients samples or control samples (“GSE33814”);
(5) GEO accession number GSE160016 that provides expression patterns of genes that are differentially expressed in the liver of NAFLD patients compared to control subjects (“NAFLD”); and
([6) GEO accession number GSE105127 that provides expression patterns of genes that are differentially expressed in the liver of steatosis patients compared to non-obese and obese control subjects (“Steatosis”).

The heatmap is limited to the 48 genes that were designated to be differentially expressed compared to the respective controls in each of the following four sets: (1) the three liver spheroid disease states, (2) the GSE33814 data set, (3) the GSE16001 data set, and (4) the GSE105127 data set. The differential gene expression pattern of the NASH induced liver spheroids was determined to more closely resemble the differential gene expression pattern seen in the liver of steatohepatitis patients (GSE33814) that the differential gene expression pattern seen in the liver of NALFD patients (GSE16001) or of steatosis patients (GSE105127). In addition, out of the three liver spheroid disease states (NASH, early-stage fibrosis and late-stage fibrosis), the differential gene expression pattern of the NASH induced liver spheroids was determined to most closely resemble the differential gene expression pattern seen in the liver of steatohepatitis patients (GSE33814).

The results shown in FIGS. 11A-11B demonstrates that the induction of NASH through a sequential treatment of free fatty acids and TGF-β1 in bi-component (PHH+HSC) liver spheroids is a notably good model for clinical steatohepatitis (NASH) that demonstrated comparable patterns of gene expression changes.

5.2. Enriched Biological Processes from Differentially Expressed Genes FFA+TGF-β1 Treated Groups Compared to Healthy Control Group:

As explained in the paragraphs hereinabove, total RNA was isolated from Liver organoid samples. Agilent based arrays (SurePrint G3 Human CGH Microarray 8×60K) was used for the Microarray hybridization. The raw count files (output from Microarray) were used for the downstream bioinformatics analysis.

Post normalization, array control probes were filtered out. In the case of multiple probes present for a single gene, only the highest expressing probe values were further taken. Post this step, the differences between the number of probes and number of genes negated out. This follows with a filtration step where genes that expressed above the background expression values were taken further for downstream applications. The final Normalized expression values of 19187 genes were later used for calculating Differential expression between Diseased vs Healthy control samples.

For obtaining Differential expression, normalized (Log 2) expression values of healthy control organoids were subtracted from normalized (Log 2) expression values of Diseased (TGF-β1+FFA Treated) organoids. The Log 2 Fold Changes obtained between Diseased vs Healthy, were further filtered on an arbitrary cut-off value of “1” (for Up regulated genes in Diseased) and “−1” (for Down regulated genes in Diseased) to get only the highly differentially expressed gene list (n=1883 genes).

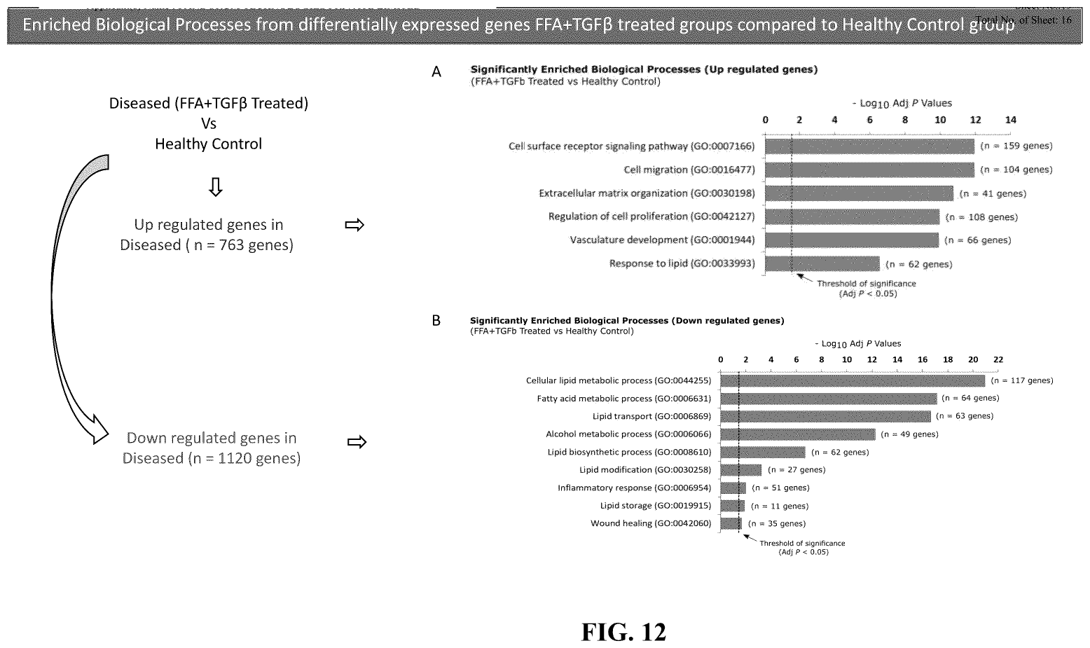

As observed in FIG. 12, there are 763 Up regulated genes and 1120 down regulated genes in the Diseased sample, compared to the Healthy counterpart.

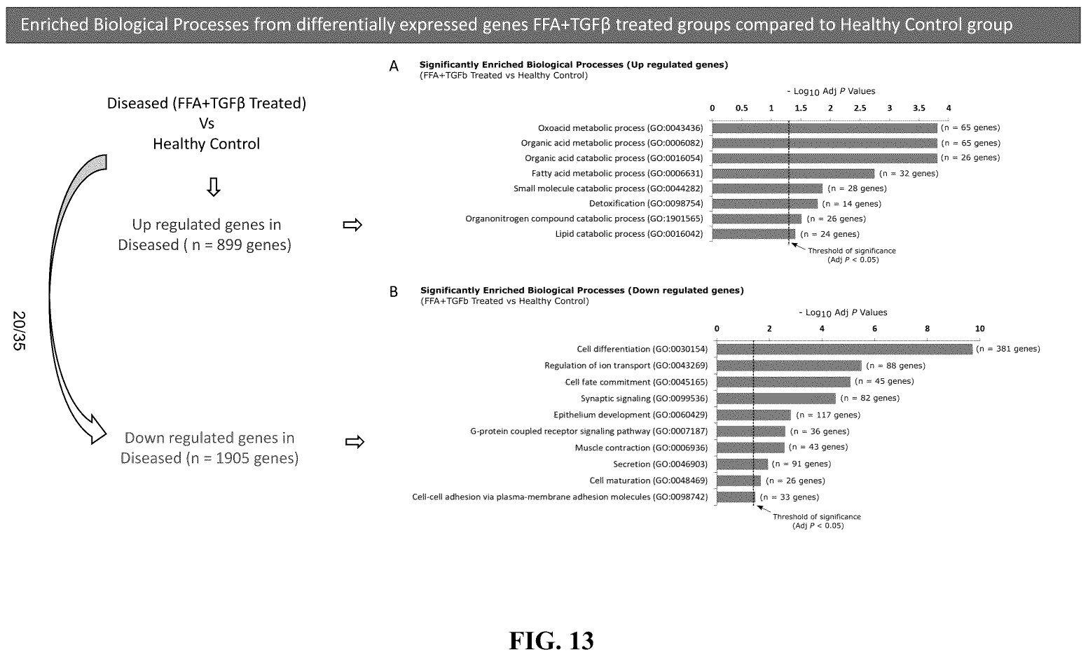

Similarly, FIG. 13 shows 899 Up regulated genes and 1905 down regulated genes in the Diseased sample, compared to the Healthy counterpart.

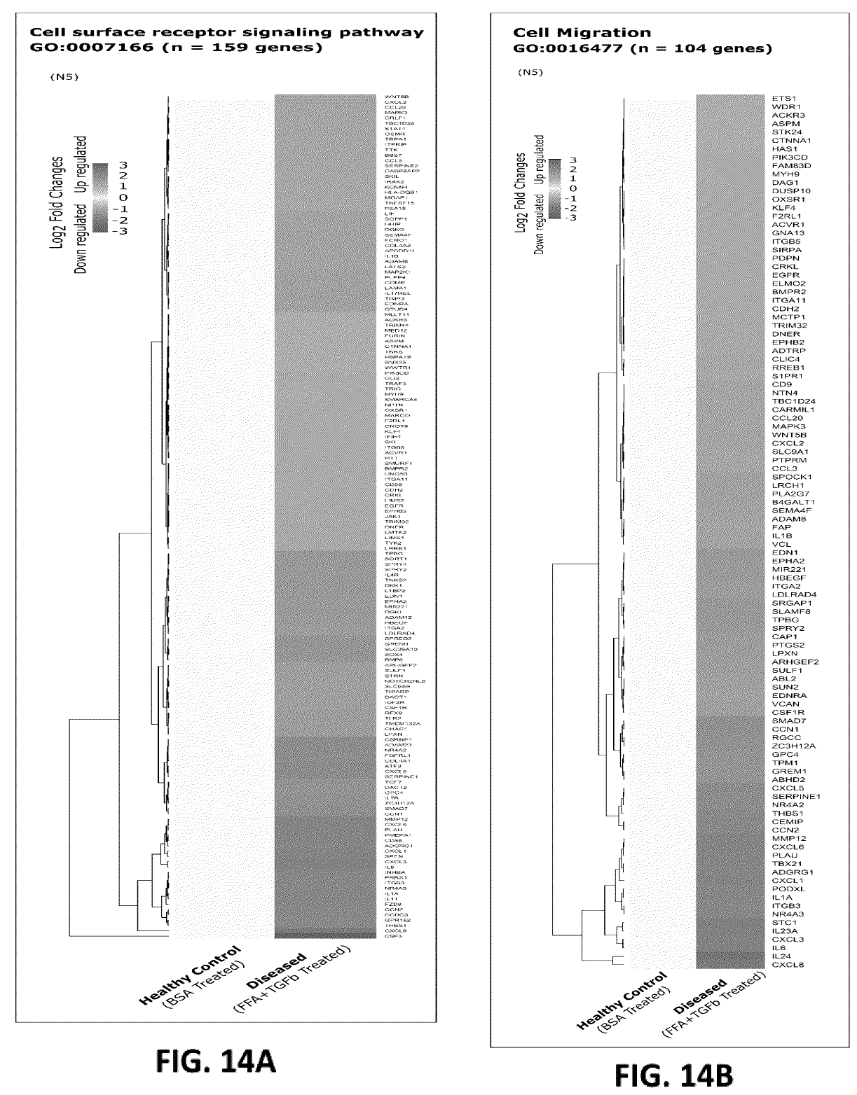

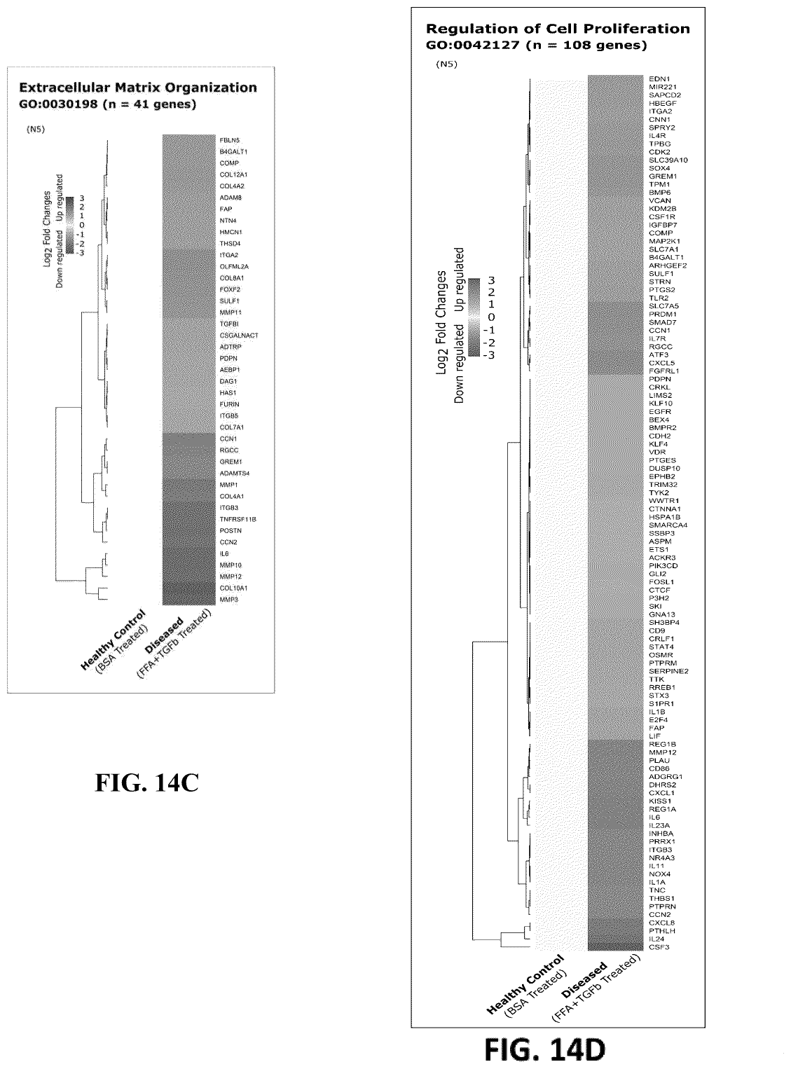

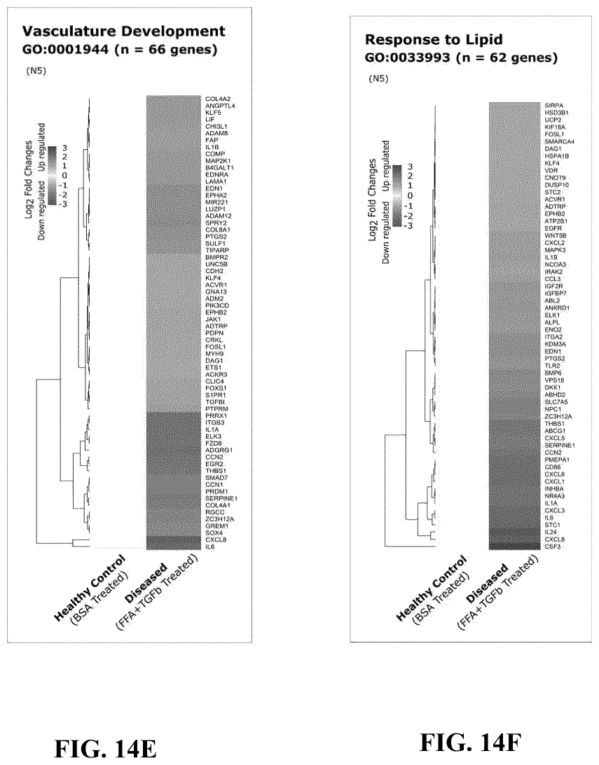

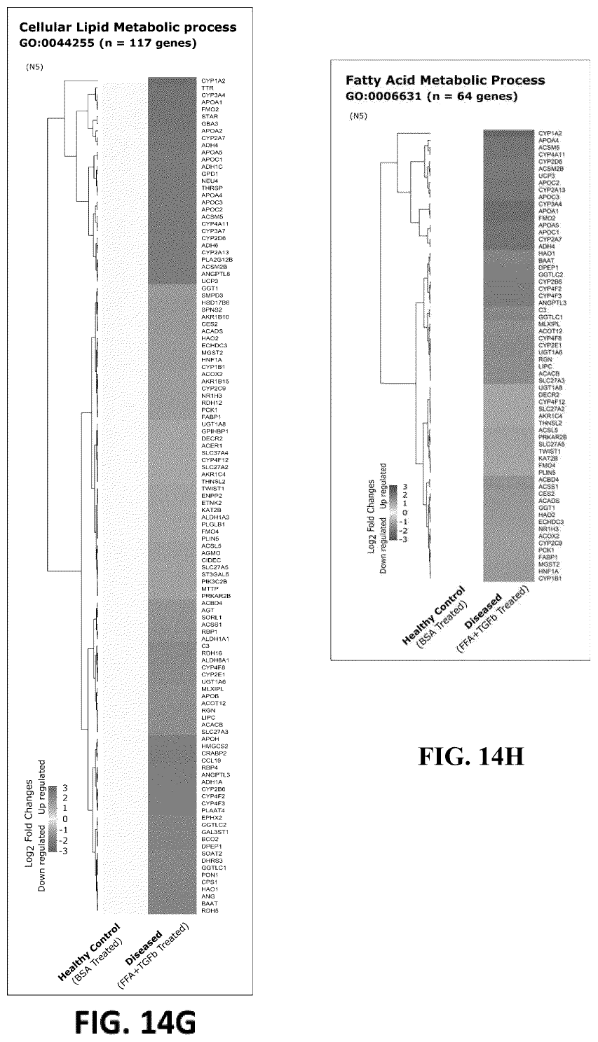

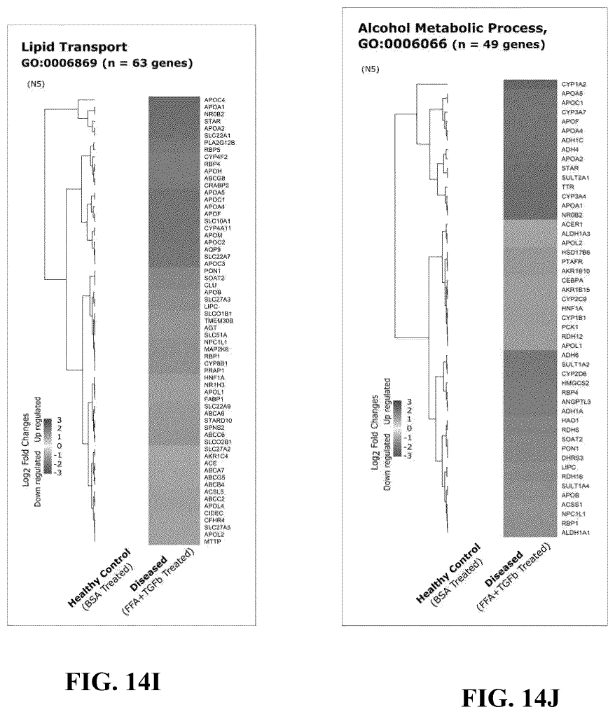

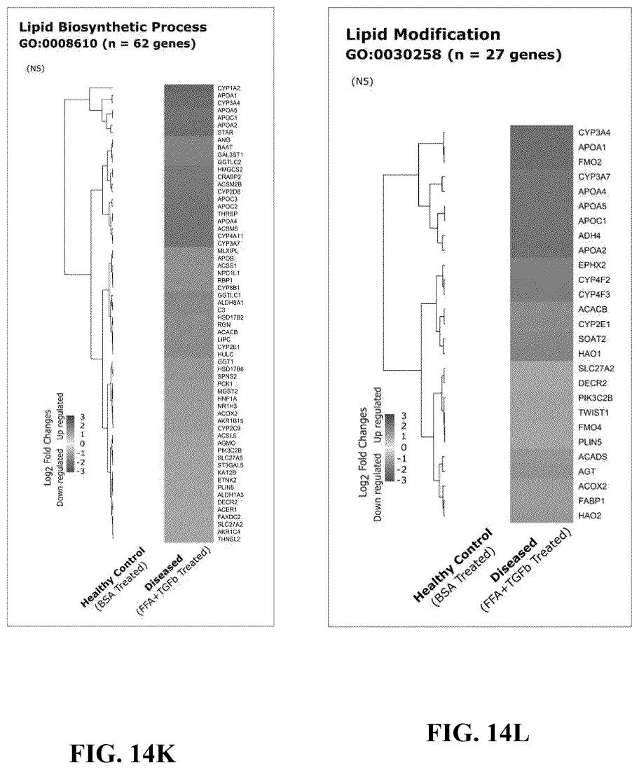

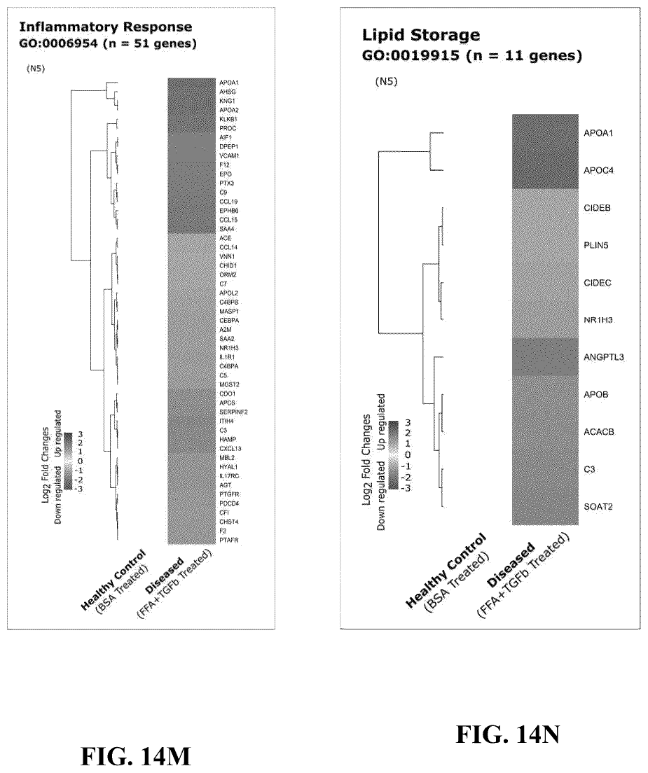

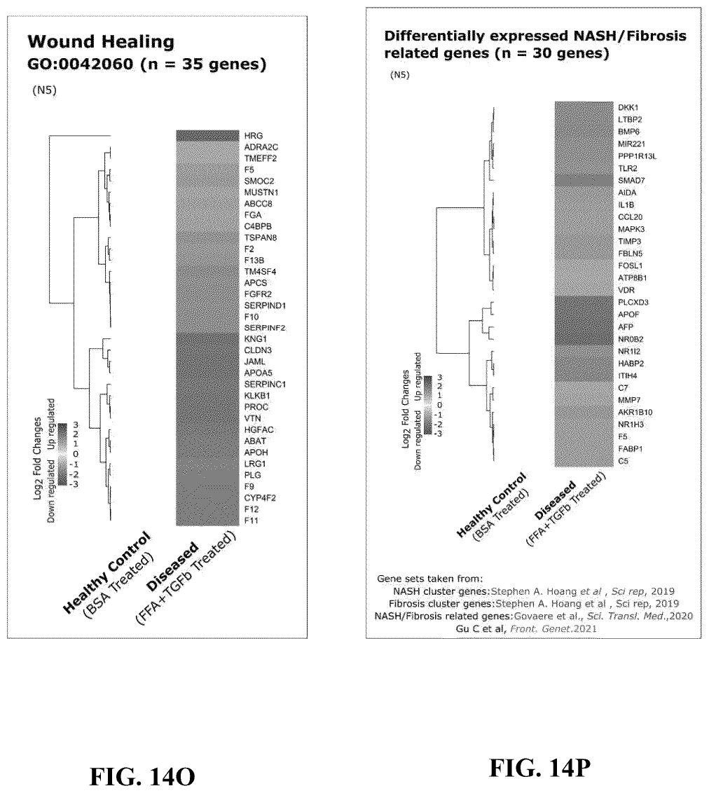

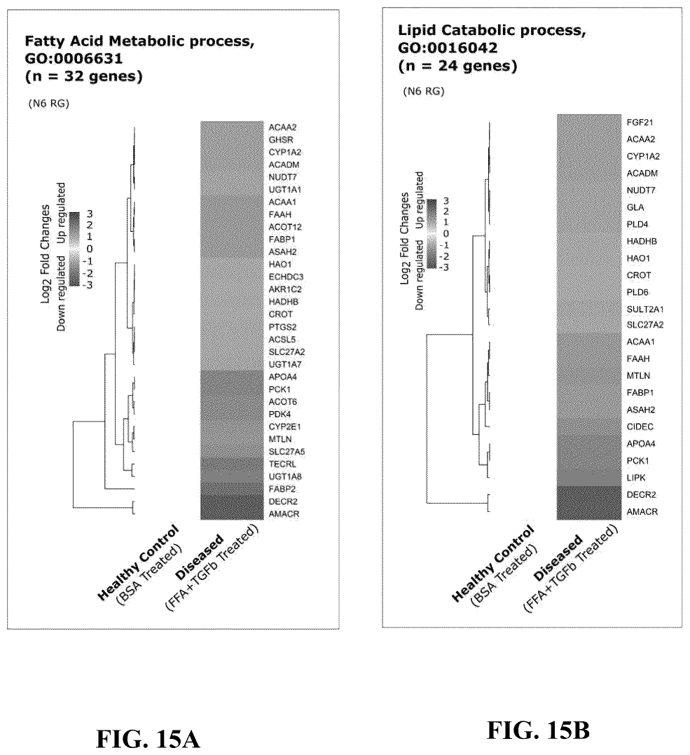

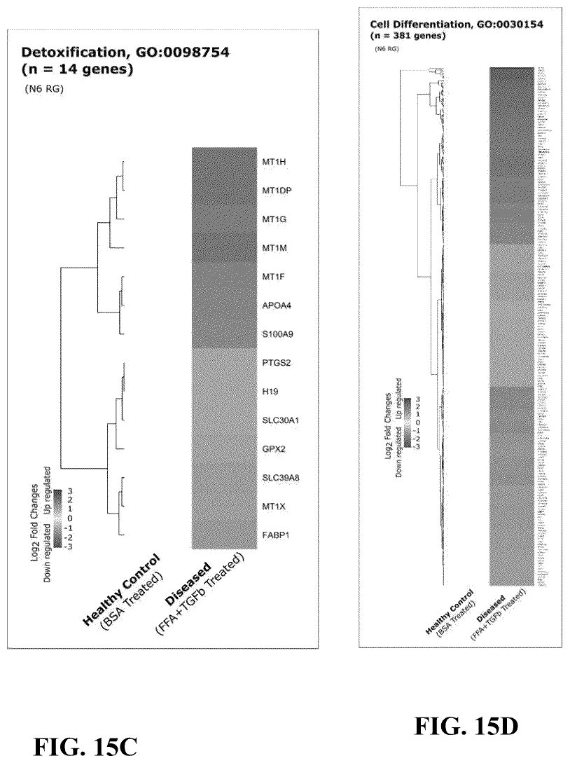

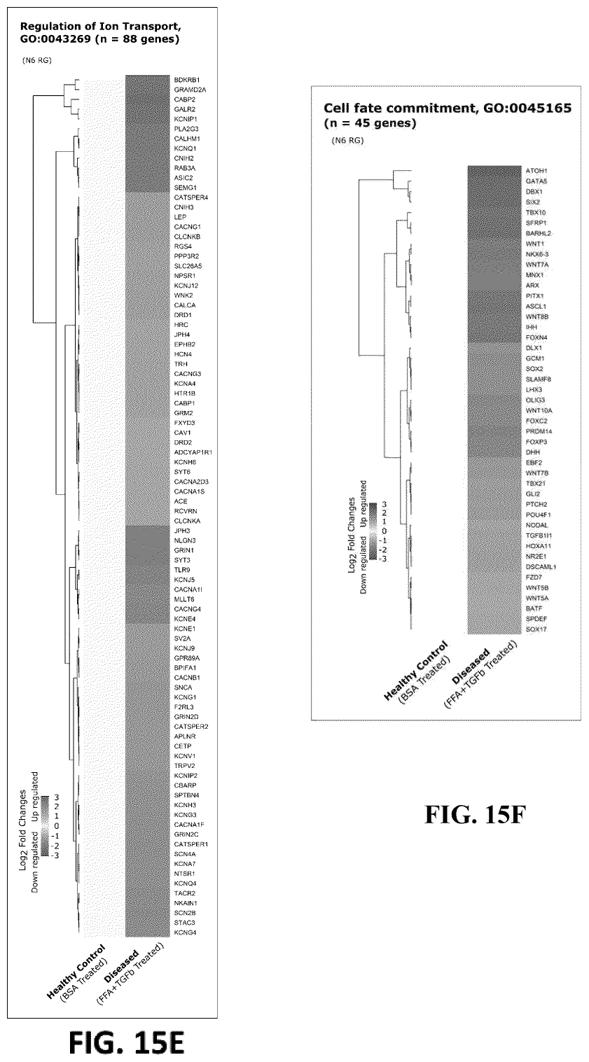

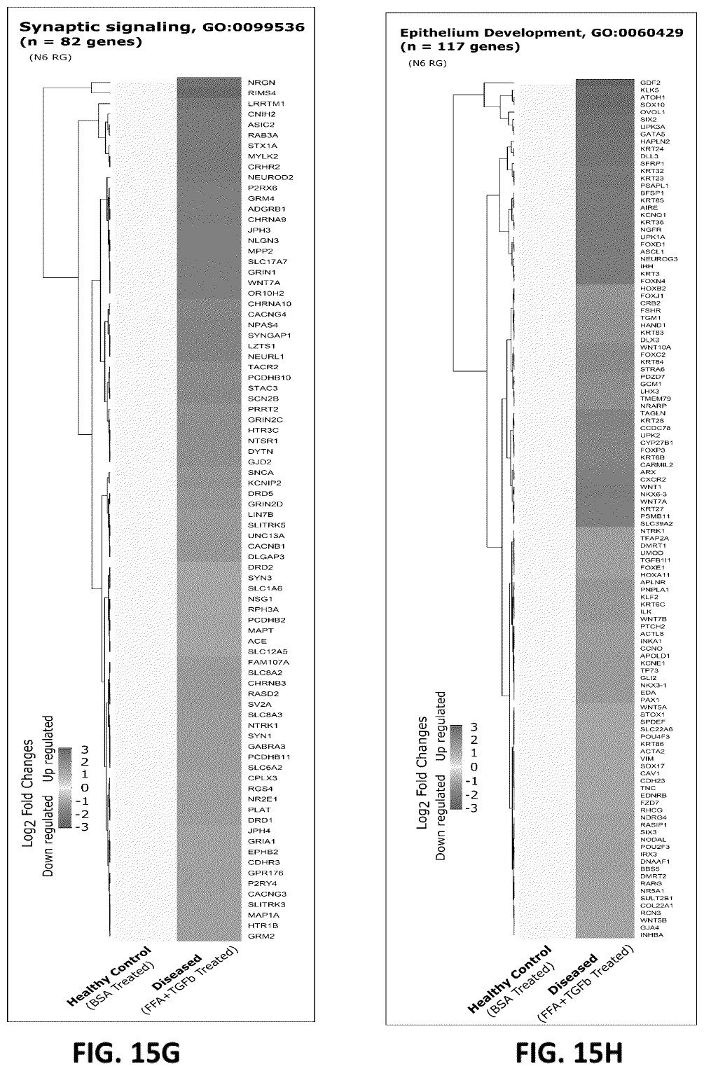

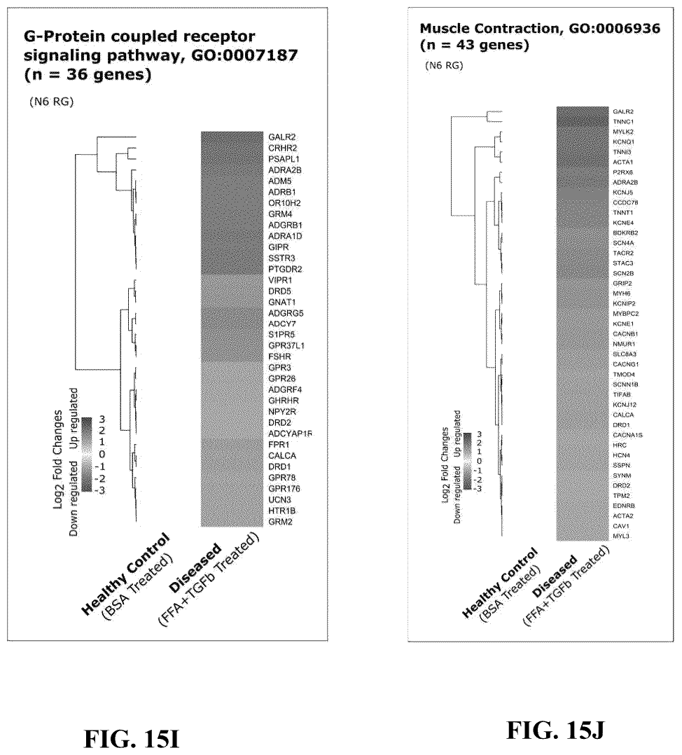

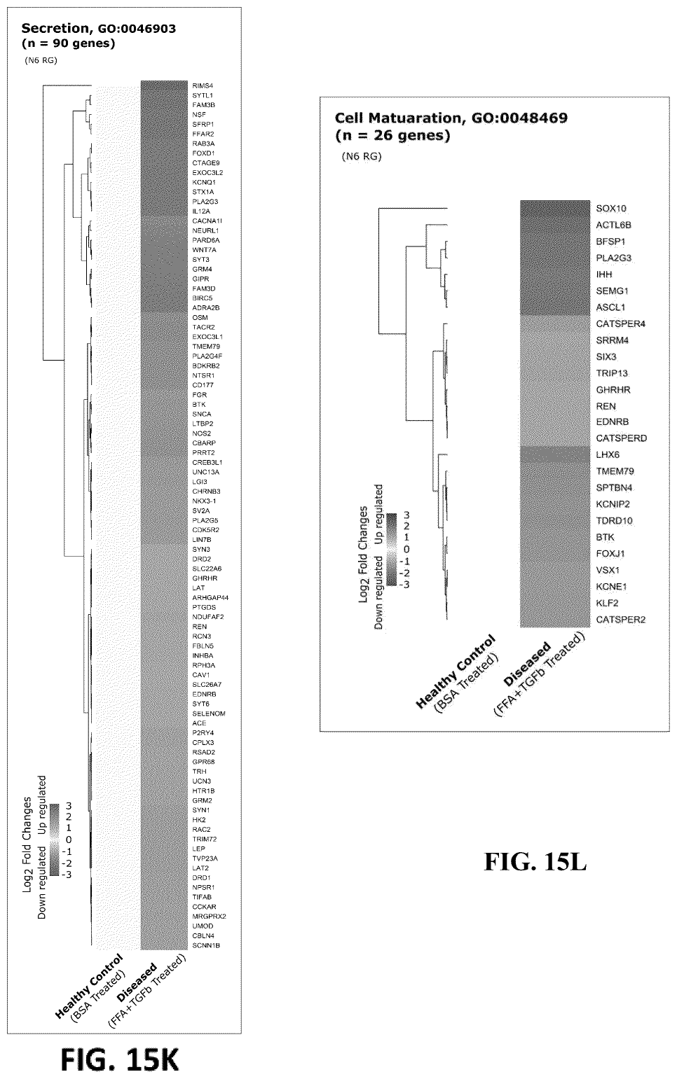

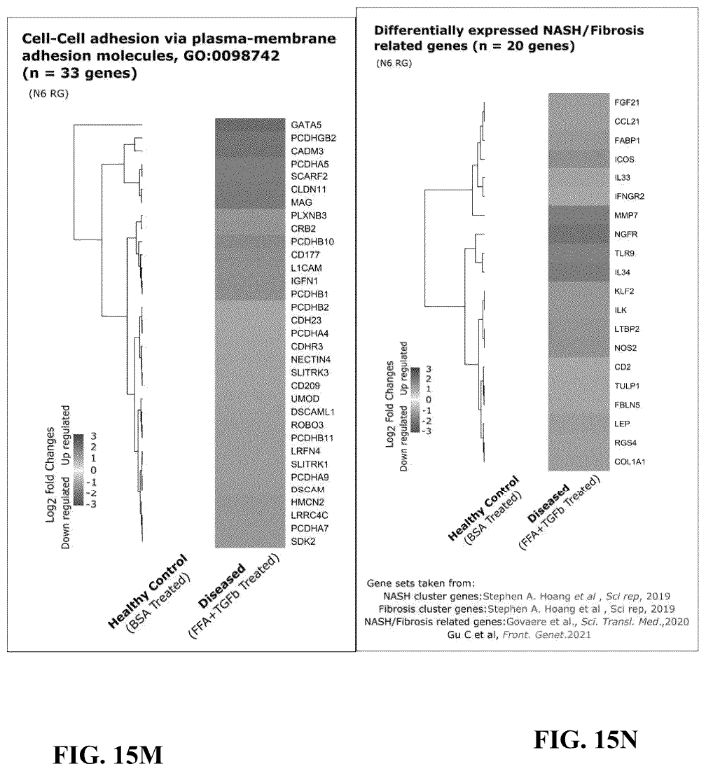

Subsequently, these gene sets were individually used for Gene Ontology enrichment analysis through “DAVID” (B. T. Sherman et al, Nucleic Acids Research, 2022. doi:10.1093/nar/gkac194). Pathways that are enriched (FDR corrected p value<0.05) are further reported through bar-plot representation. Constituent genes from the key (relevant) enriched pathways were further represented through expression heatmaps (FIG. 14 (A to P) and FIG. 15 (A to N) corresponding to the biological processes noted in FIGS. 12 and 13 respectively).

A list of important genes either upregulated or downregulated in FFA+TGF-β1 treated groups compared to Healthy Control group in different biological process is provided below:
Extracellular matrix organization (GO:0030198): A process that is carried out at the cellular level which results in the assembly, arrangement of constituent parts, or disassembly of an extracellular matrix. The extracellular matrix (ECM) is a critical component of the human liver microenvironment. Forming a fibrous scaffold, the ECM provides a surface for cell adhesion, space for cell growth and migration, and acts as reservoir for signaling molecules.

TABLE 2

| | Protein | Gene | |
|---|---|---|---|
| 1 | Collagen type IV alpha 1 chain | COL4A1 | Up regulated |
| 2 | Matrix metalloproteinase-12 | MMP12 | Up regulated |
| 3 | matrix metalloproteinase-3 | MMP3 | Up regulated |
| 4 | matrix metalloproteinase-10 | MMP10 | Up regulated |
| 5 | matrix metalloproteinase-11 | MMP11 | Up regulated |
| 6 | matrix metalloproteinase-9 | MMP9 | Up regulated |
| 7 | Fibrinogen beta chain | FGB | Up regulated |
| 8 | Fibronectin | FN1 | Up regulated |
| 9 | Fibulin-5 | FBLN5 | Up regulated |
| 10 | Integrin alpha-2/CD49b | ITGA2 | Up regulated |
| 11 | Platelet-derived growth factor | PDGF | |
| 12 | TIMP metallopeptidase inhibitor 1 | TIMP1 | Down |
| | | | regulated |
| 13 | Tissue inhibitor of metalloproteinases | TIMP2 | Up regulated |
| | 2 | | |
| 14 | Alpha smooth muscle actin | ACTA2 | Up regulated |
| 15 | Interleukin-33 | IL33 | Up regulated |

Xenobiotic metabolic process (GO:0017144): The chemical reactions and pathways involving a xenobiotic compound, a compound foreign to the organism exposed to it. It may be synthesized by another organism (like ampicillin) or it can be a synthetic chemical.

TABLE 3

| | Protein | Gene | |
|---|---|---|---|
| 1 | Flavin-containing monooxygenase 5 | FMO5 | Down regulated |
| 2 | UDP-glucuronosyltransferase 2B7 | UGT2B7 | Down regulated |
| 3 | Sulfotransferase 2A1 | SULT2A1 | Down regulated |
| 4 | Glutathione S-transferase A5 | GSTA5 | Down regulated |
| 5 | Flavin-containing monooxygenase 2 | FMO2 | Down regulated |
| 6 | Nuclear receptor ROR-gamma | RORC | Down regulated |
| 7 | ATP-binding cassette sub-family C | ABCC4 | Up regulated |
| | member 4 | | |
| 8 | Nuclear receptor subfamily 1 group I | NR1I2 | Down regulated |
| | member 2 | | |
| 9 | Cocaine esterase | CES2 | Down regulated |
| 10 | Glutathione S-transferase A2 | GSTA2 | Down regulated |

Cellular lipid metabolic process Gene Ontology Term/Lipid Metabolism (GO:0044255): The chemical reactions and pathways involving lipids, as carried out by individual cells.

TABLE 4

| | Protein | Gene | |
|---|---|---|---|
| 1 | Cytochrome P450 2A7 | CYP2A7 | Down regulated |
| 2 | Cytochrome P450 2B6 | CYP2B6 | Down regulated |
| 3 | Cytochrome P450 4F2 | CYP4F2 | Down regulated |
| 4 | Apolipoprotein B-100 | APOB | Down regulated |
| 5 | All-trans-retinol dehydrogenase | ADH4 | Down regulated |
| | [NAD(+)] ADH4 | | |
| 6 | Apolipoprotein A-I | APOA1 | Down regulated |
| 7 | Carbohydrate-responsive element- | MLXIPL | Down regulated |
| | binding protein | | |
| 8 | Cytochrome P450 2D6 | CYP2D6 | Down regulated |
| 9 | Cytochrome P450 2E1 | CYP2E1 | Down regulated |
| 10 | Cytochrome P450 3A4 | CYP3A4 | Down regulated |

Tissue Remodeling (GO:0048771); The reorganization or renovation of existing tissues. This process can either change the characteristics of a tissue such as in blood vessel remodelling or result in the dynamic equilibrium of a tissue such as in bone remodelling.

TABLE 5

| | Protein | Gene | |
|---|---|---|---|
| 1 | Interleukin-23 subunit alpha | IL23A | Up regulated |
| 2 | Interleukin-1 alpha | IL1A | Up regulated |
| 3 | NADPH oxidase 4 | NOX4 | Up regulated |
| 4 | Ephrin type-B receptor 2 | EPHB2 | Up regulated |
| 5 | Plasminogen | PLG | Down regulated |
| 6 | Osteopontin | SPP1 | Down regulated |
| 7 | Fibulin-5 | FBLN5 | Up regulated- |
| 8 | Thrombospondin-2 | THBS2 | Up regulated- |
| 9 | Collagen alpha-2(I) chain | Col1A2 | Up regulated- |

1. A method of inducing non-alcoholic steatohepatitis within a multi-component liver spheroid, comprising:
a) providing two or more distinct liver cell types;
b) seeding a mixture of the at least two or more distinct liver cell types to generate a multi-component liver spheroid;
c) inducing steatosis in the multi-component liver spheroid through treatment of one or more steatosis inducers;
d) inducing steatohepatitis in the multi-component liver spheroid through a combinatorial treatment of a mixture of the one or more steatosis inducers and one or more fibrosis inducers; and
e) inducing fibrosis in the multi-component liver spheroid through treatment with one or more fibrosis inducers.

2. The method of claim 1, wherein the at least two or more distinct liver cell types are primary liver cells.

3. The method of claim 1, wherein the multi-component liver spheroid is a bi-component liver spheroid consisting of a first type of liver cell and a second type of liver cell.

4. The method of claim 3, wherein the first type of liver cell is hepatocytes, and the second type of liver cell is hepatic stellate cells.

5. The method of claim 1, wherein the multi-component liver spheroid is comprised of three or more types of liver cells selected from the group consisting of hepatocytes, hepatic stellate cells, Kupffer cells, and liver endothelial cells.

6. The method of claim 3, wherein the first type of liver cell and the second type of liver cell are seeded at a cell count ratio within the range of about 50:50 to about 90:10.

7. The method of claim 6, wherein the cell count ratio of the first type of liver cell to the second type of liver is about 70:30.

8. (canceled)

9. The method of claim 1, wherein the one or more steatosis inducers comprises one or more free fatty acids.

10. The method of claim 9, wherein the one or more free fatty acids comprises one or more free fatty acids selected from the group consisting of oleic acid, palmitic acid, stearic acid, lineolenic acid, and linoleic acid.

11. The method of claim 9, wherein the one or more free fatty acids comprises a mixture of two or more free fatty acids.

12. The method of claim 11, wherein the mixture of two or more free fatty acids includes a first free fatty acid and a second free fatty acid at a ratio by weight of about 2:1.

13. The method of claim 11, wherein the first fatty acid is oleic acid, and the second fatty acid is palmitic acid.

14. (canceled)

15. The method of claim of claim 9, wherein the multicomponent liver spheroids are treated with the one or more steatosis inducers for about 5 days to about 7 days to induce steatosis.

16. The method of claim 1, wherein the one or more fibrosis inducers includes one or more inflammatory cytokines.

17. The method of claim 16, wherein the one or more inflammatory cytokines are selected from the group consisting of: TGF-β1, IL-1β, TNFα, IL-6, IL-15, IL-17, and IL-18, preferably TGF-β1.

18. (canceled)

19. The method of claim 1, wherein inducing steatohepatitis in the liver spheroids includes treating the liver spheroid with the mixture of the one or more steatosis inducers and one or more fibrosis inducers for about 24 hours to about 72 hours, preferably for about 48 hours.

20. The method of claim 17, wherein inducing fibrosis in the liver spheroid includes removing the one or more steatosis inducers and continuing treatment of the multi-component liver spheroid with the one or more fibrosis inducers for about another 24 hours to about 72 hours, preferably about another 48 hours.

21. A multi-component liver spheroid produced with the method of claim 1.

22. A bi-component liver spheroid produced with the method of claim 1, wherein the two or more distinct liver cell types consists of the hepatocytes, and the hepatic stellate cells.

23. A multi-component steatohepatic liver spheroid that is characterized by having a transcriptome profile characterized by one or more of, as compared to a healthy liver spheroid:
(a) at least a two-log fold reduction in MT3;
(b) at least a two-log fold reduction in APOA4; and
(c) at least a two-log fold reduction in IGFBP1.
